(19) United States
(12) Patent Application Publication
(10) Pub. No.: US 2012/0052059 A1
(43) Pub. Date: Mar. 1, 2012

(54) REGULATORY B CELLS AND THEIR USE

(75) Inventors: David Rothstein, Pittsburgh, PA (US); Qing Ding, Pittsburgh, PA (US)

(73) Assignee: University of Pittsburgh - Of the Commonwealth System of Higher Education

(21) Appl. No.: 13/223,169

(22) Filed: Aug. 31, 2011

Related U.S. Application Data
(60) Provisional application No. 61/379,302, filed on Sep. 1, 2010.

(51) Int. Cl. A61K 39/395; A61K 35/12; G01N 33/53; A61P 35/00; C12Q 1/02; C12N 5/0781; A61P 37/02

(52) U.S. Cl. 424/131.1; 435/326; 424/93.71; 435/29; 435/7.1

(57) ABSTRACT

Isolated regulatory B cells are disclosed, and compositions including these isolated regulatory B cells. The isolated regulatory B cells are mammalian and express T cell immunoglobulin mucin-1 (TIM-1). In some embodiments the regulatory B cells produce IL-10. Methods for treating a subject with an immune-mediated disorder are disclosed. These methods include administering to the subject a therapeutically effective amount of a composition including regulatory B cells, thereby treating the immune mediated disorder in the subject. Methods are also disclosed for treating a subject with an immune-mediated disorder, wherein the methods include administering to the subject a therapeutically effective amount of an antibody that specifically binds TIM-1, wherein the antibody is activates TIM-1⁺CD19⁺ B cells. Methods are also disclosed for assessing the immune status of a subject.

### PRIORITY CLAIM

This claims the benefit of U.S. Provisional Application No. 61/379,302, filed Sep. 1, 2010, which is incorporated by reference herein.

### STATEMENT OF GOVERNMENT SUPPORT

This invention was made with United States government support pursuant to grant AI070820 from the National Institutes of Health; the United States government has certain rights in the invention.

### FIELD

This relates to the field of immunology, specifically to regulatory B cells and method for modifying regulatory B cell activity.

### BACKGROUND

In addition to humoral immunity, B cells play an increasingly recognized role in shaping T effector cell responses through Ag presentation, costimulation, and cytokine production (Yanaba et al., Immunol Rev 223, 284-299, 2008). The importance of B cells to cellular immunity is underscored by studies in both humans and mice showing that B cell deficiency or depletion can ameliorate autoimmune diseases primarily mediated by T cells, including type I diabetes (Hu et al., J Clin Invest 117, 3857-3867, 2007), and rheumatoid and collagen-induced arthritis (Bouaziz et al., Proc Natl Acad Sci USA 104, 20878-20883, 2007; Edwards et al., N Engl J Med 350, 2572-2581, 2004). However, in a variety of other murine models, including EAE (a model for multiple sclerosis), (Fillatreau et al., Nat Immunol 3, 944-950, 2002; Matsushita et al., J Clin Invest 118, 3420-3430, 2008), inflammatory bowel disease (IBD) (Mizoguchi et al., Immunity 16, 219-230, 2002), and allergic skin reactions (contact hypersensitivity) (Watanabe et al., Am J Pathol 171, 560-570, 2007; Yanaba et al., Immunity 28, 639-650, 2008), B cell deficiency or depletion, worsens disease, suggesting that B cells can also exhibit inhibitory function. Indeed, subpopulations of splenic B cells from naïve or autoimmune mice have been shown to exhibit regulatory activity and inhibit inflammation in an IL-10-dependent manner (Dilillo et al., Ann NY Acad Sci 1183, 38-572010; Mauri and Ehrenstein, Trends Immunol 29, 34-40, 2008; Mizoguchi and Bhan, J Immunol 176, 705-710, 2006; Yanaba et al., Immunol Rev 223, 284-299, 2008).

However, definitive identification is challenging because regulatory B cells (Breg) lack a specific marker and interleukin (IL)-10 expression has only been detected ex vivo. Breg frequently express markers characteristic of marginal zone (MZ) (Brummel and Lenert, J Immunol 174, 2429-2434, 2005; Gray et al., Proc Natl Acad Sci USA 104, 14080-14085, 2007; Lenert et al., J Clin Immunol 25, 29-40, 2005) or less mature transitional-2 MZ precursors (T2-MZ) (Evans et al., J Immunol 178, 7868-7878, 2007; Mizoguchi et al., Immunity 16, 219-230, 2002). However, in some studies, Breg have been found to reside within the much larger follicular (FO) B cell subset (Evans et al., supra, 2007; Gray et al., supra, 2007). Thus, IL-10 expressing cells with Breg activity are distributed amongst non-regulatory B cells and are widely distributed amongst various phenotypic B cell subsets. However, since only around 1% of total splenic B cells are capable of IL-10 production, and these regulatory B cells have no specific marker, they are hard to identify or separate from the vast majority of non-regulatory B cells.

Recently, Yanaba et al identified a subset of splenic CD1d^{Hi}CD5⁺ B cells that inhibit contact hypersensitivity upon adoptive transfer (Yanaba et al., supra, 2008). These Breg comprise a small proportion (−2%) of the overall splenic B cell population, but are highly enriched for cells expressing IL-10 (9-15%) compared to other B cell subsets. The CD1d^{Hi}CD5⁺ phenotype partially overlaps with that of marginal zone (MZ) and T2-MZ B cells and it has been postulated that the CD1d^{Hi}CD5⁺ population may account for most Breg activity observed in spleen. On a numerical basis, despite a low frequency (about 1%) more IL-10+ B cells are contained in the 97% of non-CD1d^{Hi}CD5+ B cells. Direct examination reveals amongst the total B cells expressing IL-10, ≦20% fall into the CD1dHiCD5+ subset. Thus, CD1d and CD5 are not ideal markers for identification or isolation of regulatory B cells. A need remains for other markers and methods to identify Breg. The identification of these Breg markers means that isolated populations of Bregs can be produced and used in a number of methods.

### SUMMARY

Isolated regulatory B cells are disclosed, and compositions including these isolated regulatory B cells. The isolated regulatory B cells are mammalian and express T cell immunoglobulin mucin-1(TIM-1). In some embodiments the regulatory B cells are TIM-1⁺CD19⁺. In additional embodiments, the cells express interleukin-10.

In some embodiments, methods for treating a subject with an immune-mediated disorder are provided. These methods include administering to the subject a therapeutically effective amount of a composition including regulatory B cells, thereby treating the immune mediated disorder in the subject.

In additional embodiments, methods are provided for treating a subject with an immune-mediated disorder, wherein the methods include administering to the subject a therapeutically effective amount of an antibody that specifically binds TIM-1, wherein the antibody induces regulatory B cells by increasing the number or activity of TIM-1⁺CD19⁺ B cells. In some examples, the methods include measuring TIM-1⁺CD19⁺ B cells.

In further embodiments, methods are provided for inducing an immune response to a tumor antigen in a subject with a tumor. These methods include decreasing the number of TIM-1⁺CD19⁺ regulatory B cells in the subject and administering the tumor antigen or a nucleic acid encoding the tumor antigen to the subject.

Methods are also disclosed for assessing the immune status of a subject. These methods include selecting a subject with an immune-mediated disorder or suspected of having an immune-mediated disorder; and detecting the number of CD19⁺TIM-1⁺ regulatory B cells in an initial biological sample from the subject.

The foregoing and other features and advantages will become more apparent from the following detailed description of several embodiments, which proceeds with reference to the accompanying figures.

### BRIEF DESCRIPTION OF THE FIGURES

FIGS. 1A-1C. B lymphocytes express high levels of TIM-1 and are required for anti-TIM-1-mediated prolongation of allograft survival. FIG. 1A is a representative flow cytometry plots showing TIM-1 expression on splenic CD4+ and CD8+ T cells and on CD19+ B cells, in naïve BALB/c mice (top row), or 14 days after immunization with either allogeneic (C57BL/6) islets (middle row) or ovalbumin (Ova; 20 μg with 4 mg alum intraperitoneally (i.p.) on days 0 and 7; bottom row). Numbers in each plot are the percent of TIM-1+ cells within each population. Representative of more than three mice/group. FIG. 1B are Kaplan-Meir plots of graft survival of wild-type (WT) animals with and without B-depletion. Chemically diabetic BALB/c mice were untreated or subjected to B cell depletion with anti-CD20 (250 ug i.v. on days −14 and −1) followed by transplantation with C57BL/6 islets. Allograft recipients were either untreated or treated with anti-TIM-1 (RMT1-10; 0.5 mg d-1, and 0.3 mg day (d) 0 and 5). ̂ p<0.01 α-anti-Tim-1 vs. control; ̂̂ p<0.05 α-CD20+α-TIM-1 vs. α-CD20. FIG. 1C are Kaplan-Meir plots of graft survival of B-deficient animals, with and without B cell reconstitution. Chemically diabetic B-deficient JHD mice (BALB/c) were unmanipulated or received 10⁷ wt syngeneic B cells followed by transplantation with C57BL/6 islets (d0). Allograft recipients were either untreated or treated with anti-TIM-1 (as above). Ŝ p<0.05 α-TIM-1 vs. control; ̂̂ p<0.01 B+α-Tim-1 vs. B.

FIGS. 2A-2C. B lymphocytes are required for Th2-deviation induced by α-TIM-1. Chemically diabetic BALB/c mice expressing an IL-4-EGFP reporter (4get mice) were untreated or subjected to B cell depletion with anti-CD20 followed by transplantation with C57BL/6 islets. Allograft recipients were either untreated or treated with anti-TIM-1 (as in FIG. 1B). Cytokine expression on splenic CD4⁺ T cells was examined on day 14 after transplantation by flow cytometry after in vitro stimulation (see Methods) and surface staining for CD4. IL-4 was detected by EGFP expression, and IFN-γ, IL-10 and Foxp3, by intracellular staining. FIG. 2A is a set of plots showing IL-4, IFN-γ and IL-10 expression on CD4⁺ T cells by flow cytometry. The percent of CD4 cells expressing cytokines are shown in each plot. Data are representative of five or more mice/group. FIG. 2B shows the frequency (mean+SD) of CD4⁺ T cells expressing IFN-γ, IL4 or IL-10 in each treatment group. (n≧5 mice/group). * p<0.05 vs. Txpl untreated; **p<0.05 vs. Txpl +α-CD20; *** p<0.05 vs. Txpl +α-TIM-1. FIG. 2C shows representative Foxp3 and IL-10 expression by CD4⁺ T cells in each treatment group by flow cytometry. (n=3 mice/group).

FIG. 3A-3E. B cell IL-4 and IL-10 are primarily expressed by TIM-1⁺ B cells: induction by TIM-1 ligation. FIG. 3A is a set of plots showing IL-4 expression (left panel) and IL-10 expression (right panel) by flow cytometry on splenic B cells from naive BALB/c (4get) mice, and untreated (Txpl) or anti-TIM-1 treated (Txpl+α-TIM-1) BALB/c (4Get) recipients of C57BL/6 islets (day 14). Plots are gated on CD19+ B cells (“control” and “total B cells”), and on TIM-1⁻ and TIM-1⁺ CD19+ B cells, as indicated. IL-4 was determined by EGFP expression using IL-4 reporter (4get) mice, and IL-10 was determined by intracellular staining. Fluorescence controls (control) used wt littermates for EGFP and isotype control antibodies for intracellular staining. Numbers in each plot indicate the percent of gated cells expressing the indicated cytokine. Data representative of five or more mice per group. FIG. 3B shows the average frequency of CD19⁺ B cells expressing IFN-γ, IL-4 or IL-10, from mice as described in 4A. Data are expressed as the mean+SD of the values from five mice per group. * p<0.05 vs. naïve; ** p<0.05 vs. Txpl. FIG. 3C shows the average frequency of TIM-1− or TIM-1⁺ B cells from anti-TIM-1-treated BALB/c recipients of C57BL/6 islet allografts (day 14), expressing IL4 (EGFP), or IL-10 or IFN-γ (intracellular immunofluorescence). Data are the mean+SD from five individual mice. * p<0.01 vs. TIM-1⁻ B. FIG. 3D shows TIM-1 expression on splenic B cells, or CD4 and CD8 T cells from naïve BALB/c mice or untreated (Txpl) or anti-TIM-1 treated (Txpl+α-TIM-1) BALB/c recipients of C57BL/6 islets (day 14). Data are the mean+SD from 5 individual mice. * p<0.05 vs. naïve. FIG. 3E is a set of plots. Sorted TIM-1⁻ B cells from naïve BALB/c mice were adoptively transferred into JHD islet allograft recipients with or without anti-TIM-1 treatment. Representative Tim-1 expression by flow cytometry on sorted TIM-1− B cells before transfer and those recovered from spleen 14 d later. Gray fill, isotype control; Black line, TIM-1 expression. Representative of three individual experiments.

FIGS. 4A-4D. B cell mediated prolongation of allograft survival are IL-10 and IL-4 dependent. FIG. 4A is a Kaplan-Meir plots of graft survival. JHD recipients of C57B/6 islet allografts were reconstituted with 10⁷ naïve B cells from wild type (WT), IL-4^{-/-}, IL-4Rα^{-/-} mice and were treated with anti-TIM-1. A p<0.01 vs. untreated JHD; AA p<0.05 vs. untreated JHD. FIG. 4B is a bar graph. Sort purified CD19+ B cells from WT, IL-4^{-/-} and IL-4Rα^{-/-} mice were adoptively transferred into JHD transplant recipients with anti-TIM-1 treatment. TIM-1 expression on transferred B cells from spleen (d 14) was detected by flow cytometry. n=3 mice per group. *p<0.01 vs. WT; ** p<0.05 vs. IL-4Rα−/−. FIG. 4C is a bar graph. IL-10 expression (intracellular staining) by B cells from (FIG. 4B), isolated 14 days after adoptive transfer into anti-TIM-1 treated allograft recipients. Representative of three mice per group. FIG. 4D is a set of plots. The upper panel is representative histograms showing TIM-1 expression by WT, IL-4^{-/-} or IL-4Rα^{-/-} TIM-1⁻ B cells after stimulation with anti-IgM (dashed line) vs. anti-IgM plus IL-4 (solid line). Isotype antibody control is shown (gray fill). Representative of 3 independent experiments. The Lower panel shows an overlay of histograms from above comparing TIM-1 expression on anti-IgM treated WT, IL-4^{-/-}, or IL-4Rα^{-/-} TIM-1⁻ B cells.

FIGS. 5A-5C. TIM-1⁺ B cells prolong allograft survival in a donor-specific and IL-10-dependent manner. FIG. 5A are Kaplan-Meir plots of graft survival. WT BALB/c recipients of C57BL/6 islets were treated with anti-TIM-1. On d14, splenic B cells were isolated and sorted into TIM1⁺ and TIM1⁻ subsets. 10⁷ TIM-1⁺ and TIM-1⁻ B cells were transferred to otherwise untreated JHD recipients of islet allografts from the same (C57BL/6) strain. ̂ p<0.01 TIM-1⁺ B vs. each other group; ̂̂ p<0.05 TIM-1⁺ B (IL-10^{-/-}) vs. untreated; TIM-1⁻ B vs. untreated, p>0.05. FIG. 5B are Kaplan-Meir plots of graft survival. TIM-1+ B from spleen of naïve BALB/c mice or from untreated BALB/c recipients of either C57BL/6 (B6) or C3H islet allografts (d14) were sort-purified and transferred (1×10⁷) into JHD recipients of B6 islets without further treatment. A p<0.01 vs. other groups. FIG. 5C are FACS polts. 10⁷ TIM-1+ or TIM-1− B cells from anti-TIM-1 treated BALB/c allograft recipients were transferred into otherwise untreated JHD IL-4-GFP reporter (JHDx4get) recipients of C57BL/6 islets on day 0.14 days later, splenic CD4⁺ T cells from JHDX4get transplant recipients that received TIM-1+, TIM-1−, or no B cells, were assessed for IL-4 production (EGFP) and IFN-γ and IL-10 (intracellular staining) by flow cytometry. Representative of three mice per group.

FIGS. 6A-6D. TIM-1⁺ Breg are highly enriched for IL-10 expression across a wide spectrum of phenotypes. Spleen cells from naïve or BALB/c or anti-TIM-1 treated BALB/c allograft recipients (d14) were activated for 5 hours in vitro (see the Example section) and assessed for expression of various cell surface markers followed by intracellular staining for IL-10 expression. All data are representative of at least three independent experiments. FIG. 6A are flow cytometry plots (left) showing CD5 vs. CD1d expression on CD19+ B cells in naïve mice and anti-TIM-1-treated allograft recipients. The CD1dHiCD5+ population is indicated in the rectangular gate (2.3-2.6% of total B cells). The main panel shows TIM-1 and IL-10 expression on CD19+(Total B), and TIM-1+ vs. TIM-1− B cells within the CD1dHiCD5+ and non-CD1dHiCD5+ B cell populations by flow cytometry. Cells from naïve mice (top two rows) and anti-TIM-1 treated allograft recipients (bottom two rows) are shown. Numbers in each plot indicate percent of gated cells expressing IL-10. FIG. 6B are representative flow cytometry plots showing expression of various markers on IL-10+CD19+ B cells. Cells within the IL-10+ B cell gate (first panel) were assessed for CD1d and CD5 expression (middle panel) or TIM-1 expression (right panel). Numbers indicate percent cells expressing indicated marker. (Representative of 6 mice). FIG. 6C is a bar graph of the percent of B cells expressing TIM-1 within follicular (FO) B cells (IgD⁺IgM⁻CD21⁺CD23⁺), marginal zone (MZ) B cells (IgM⁺IgD⁻CD21^{hi}CD23⁻), transitional 2-marginal zone (T2-MZ) precursors (IgM⁺IgD⁻CD21⁺CD23^{hi}), and transitional 1 (T1) B cells (IgM⁺IgD-CD21⁻CD23⁻) from naive and transplanted mice with or without anti-TIM-1 treatment. Data are depicted as mean (+S.D.) of 3-5 mice per group. *p<0.05 vs. Naïve; **p<0.05 Txpl+α-TIM-1 vs. Txpl. FIG. 6D is a set of bar graphs. IL-10 expression on splenic B cells within MZ, FO, T2-MZ, and T1 subsets (as in 6B) from naïve mice (Naïve) and anti-TIM-1 treated allograft recipients (Txpl +-TIM-1). B cells within each subset were gated on CD19+(Total B) cells, or TIM-1+ vs. TIM-1−CD19+ B cells. Data are depicted as mean (+S.D.) of at least three mice per group. *p<0.05, Total B: Txpl+α-TIM-1 vs. naïve; **p<0.05, TIM-1+ vs. TIM-1−.

FIG. 7 is a set of graphs showing TIM-1 expression on Th1 and Th2 after in vivo activation: Representative cytokine expression on CD4 cells (left column) and TIM-1 expression by cytokine-expressing cells (right column) determined by flow cytometry 14 days after immunization of BALB/c mice with C57BL/6 islets (Txpl) or Ovalbumin (Ova). IFNγ (allograft recipients) and IL-4 and IL-10 (Ova recipients) were determined by intracellular staining after in vitro stimulation (see the Examples section). (n=5 mice/group).

FIG. 8. Average (+SD) frequency of TIM-1+ cells amongst splenic CD19+ B cells from WT, IL-10−/−, IL-4^{-/-}, or IL-4Rα^{-/-} BALB/c mice that were naïve or recipients of C57BL/6 islet allografts with or without anti-TIM-1 treatment (d14). n=5 mice/group. * p<0.05 vs. WT.

FIG. 9. Frequency of TIM-1 expression on purified CD 19⁺TIM-1⁻ B cells after in vitro culture (48 hours) in media alone or in the presence of cytokines or B cell mitogens, as indicated. Data shown as mean values (+SD). * p<0.01 vs. medium; ** p<0.01 vs. α-CD40; *** p<0.01 vs. α-IgM. n=4 independent experiments.

FIG. 10. TIM-1+ B cells are enriched for IL-10 expression in a second mouse strain. Spleen cells from naïve IL-10 reporter mice (C57BL/6) were activated for five hours in vitro (see the Examples section). Based on cell surface marker expression, CD1d^{Hi}CD5⁺ and non-CD1d^{Hi}CD5⁺ B cell populations were further gated into TIM-1+ and TIM1− subsets and assessed for IL-10 expression based on EGFP reporter expression. WT littermates were used as fluorescence controls. Representative plots show frequencies of IL-10-expressing cells among B-cells within the indicated gates. n=3 mice FIG. 11A-11B. Expression of TIM-1 on B cells in lymph node (LN) and peritoneal cavity. FIG. 11A shows the expression of TIM-1, IL-4 and IL-10 on CD19⁺ B cells in LN. Peripheral lymph node lymphocytes from naïve BALB/c mice were activated in vitro for 5 hours (see the Examples section). CD19⁺ B cells were assessed for TIM-1 expression and cytokine expression was assessed on TIM-1⁺ and TIM-1⁻ gates. Numbers indicate percent cells in each quadrant. Data are representative of three independent experiments. FIG. 11B shows the expression of TIM-1 and IL-10 on peritoneal B1 subsets. Peritoneal washout cells from naïve BALB/c mice were activated in vitro for 5 h (see the Examples section). Cells were divided into B1a and B1b subsets based on CD5 expression. CD19+ B cells were assessed for TIM-1 expression and IL-10 expression was assessed on TIM-1+ and TIM-1− gates. Numbers indicate percent cells in each quadrant. Data are representative of five independent experiments.

FIG. 12. Human PBMC were obtained by peripheral venipuncture and density gradient centrifugation with Ficoll. Cells were incubated with fluorochrome-conjugated anti-CD19 (BD Biosciences) and anti-TIM-1 (RnD) in the presence of human IgG to block Fc receptors. Subsequently, cells were incubated with fluorochrome-conjugated anti-mouse IgG2b mAb to identify TIM-1+ B cells. As shown, approximately 22% of human (CD19+) B cells express TIM-1. These are the regulatory population of interest. Cursor placement was defined by isotype controls.

### DETAILED DESCRIPTION

The T cell Ig mucin (TIM) proteins constitute a family of costimulatory molecules that play an important role in effector differentiation of CD4 cells (Kuchroo et al., Nat Rev Immunol 8, 577-580, 2008). The eight murine and three human genes encoding TIM family members are clustered in a chromosomal region (5q32,2 in humans and 11B.1 in mice) closely associated with multiple autoimmune diseases, and polymorphic forms of TIM-1 have been associated with susceptibility to human asthma, eczema, and rheumatoid arthritis (Chae et al., Immunogenetics 56, 696-701, 2005; McIntire et al., Nat Immunol 2, 1109-1116, 2001). TIM-1 is expressed on activated T cells and is preferentially expressed on Th2 cells after polarization in vitro (Umetsu et al., Nat Immunol 6, 447-454, 2005). The major TIM-1 ligand, TIM-4, is expressed on APCs (Meyers et al., Nat Immunol 6, 455-464, 2005). TIM-1 ligation with a high affinity mAb (termed 3B3) was found to augment expansion of antigen-specific T cells (Umetsu et al., Nat Immunol 6, 447-454, 2005; Xiao et al., J Exp Med 204, 1691-1702, 2007) and promote expression of Th1 and Th17 cytokines (Xiao et al., supra, 2007), while inhibiting Treg activity and Foxp3 expression (Degauque et al., J Clin Invest 118, 735-741, 2008). Concordantly, 3B3 treatment worsens EAE (Xiao et al., supra, 2007), prevents tolerance after intra-nasal peptide administration (Umetsu et al., supra, 2005), and prevents allograft tolerance induction by anti-CD154 (Degauque et al., supra, 2008).

In contrast, a lower affinity anti-TIM-1 mAb, RMT1-10, inhibits EAE (Xiao et al., supra, 2007), prolongs cardiac allograft survival, and when combined with a short course of Rapamycin, induces long-term allograft acceptance (Ueno et al., 2008). Prolonged engraftment by RMT1-10 is dependent on inhibition of Th1 responses while preserving or promoting Th2 differentiation and Treg activity (Ueno et al., J Clin Invest 118, 742-751, 2008). These studies demonstrated that TIM-1 was a potent regulator of T cell effector responses in both auto- and allo-immunity.

It is disclosed herein that TIM-1 is a marker for B cells with regulatory activity. TIM-1 identifies regulatory B cells within each B cell subset, including but not limited to, the CD1dHi CD5+ subset. Furthermore, the use of anti-TIM-1 mAbs can specifically increase both the number and regulatory function of TIM-1+ B cells.

Results are presented that show that TIM-1 expression identifies B cells capable of IL-10 expression. In vivo, TIM-1+ B cells comprise about 8% (and up to about 25%) of total B cells that are 10-30 fold enriched for IL-10 expression compared to their TIM-1− counterparts, regardless of other phenotypic markers. Furthermore, TIM-1 is predominantly expressed on B cells rather than T cells, both constitutively and after activation. Surprisingly, the tolerogenic effects of the low affinity anti-TIM-1 mAb, RMT1-10, was completely dependent on TIM-1+ B cells. In addition, it is demonstrated that TIM-1+ regulatory B cells (Breg) are induced by TIM-1 ligation and can transfer long-term acceptance of islet allografts to otherwise untreated recipients in an IL-10 dependent fashion.

### TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Allogeneic and Autologous: Organisms, cells, tissues, organs, and the like from, or derived from, individuals of the same species, but wherein the organisms, cells, tissues, organs, and the like are genetically different one from another are “allogeneic.” Organisms, cells, tissues, organs, and the like from, or derived from, a single individual, or from a genetically identical individual are “autologous.” “Transplant rejection” refers to a partial or complete destruction of a transplanted cell, tissue, organ, or the like on or in a recipient of said transplant due to an immune response to an allogeneic graft.

Animal: Living multi-cellular vertebrate organisms, a category that includes, for example, mammals and birds. The term mammal includes both human and non-human mammals. Similarly, the term “subject” includes both human and veterinary subjects.

Autoimmune Disease: A disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

Exemplary autoimmune diseases affecting mammals include rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (for example, Crohn's disease, ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, autoimmune hemolytic anemia, pernicious anemia, and the like.

B Cell: A lymphocyte, a type of white blood cell (leukocyte), that expresses immunoglobulin on its surface and can ultimately develop into an antibody secreting a plasma cell. In one example, a B cell expresses CD19 (CD19+). An “immature B cell” is a cell that can develop into a mature B cell. Generally, pro-B cells (that express, for example, CD45 or B220) undergo immunoglobulin heavy chain rearrangement to become pro B pre B cells, and further undergo immunoglobulin light chain rearrangement to become an immature B cells. Immature B cells include T1 and T2 B cells. Thus, one example of an immature B cell is a T1 B that is an AA41^{hi}CD23¹⁰ cell. Another example of an immature B cell is a T2 B that is an AA41^{hi}CD23^{hi} cell. Thus, immature B cells include B220 expressing cells wherein the light and the heavy chain immunoglobulin genes are rearranged, and that express AA41. Immature B cells express IgM on their cell surface and can develop into mature B cells, which can express different forms of immunoglobulin (e.g., IgA, IgG). Mature B cells may also express characteristic markers such as CD21 and CD23 (e.g. CD23^{hi}CD21^{hi} cells), but do not express AA41. B cells can be activated by agents such as lippopolysaccharide (LPS), CD40 ligation, and antibodies that crosslink the B cell receptor (immunoglobulin), including antigen, or anti-Ig antibodies.

A “regulatory B cell” (Breg) is a B cell that suppresses the immune response. Regulatory B cells can suppress T cell activation either directly or indirectly, and may also suppress antigen presenting cells, other innate immune cells, or other B cells. Regulatory B cells can be CD1d^{hi}CD5⁺ or express a number of other B cell markers and/or belong to other B cell subsets. These cells can also secrete IL-10. As shown in methods, regulatory B cells also express TIM-1⁺, such as TIM-1⁺CD19⁺ B cells.

Chemotherapy; chemotherapeutic agents: As used herein, any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating neoplasms such as solid tumors. In one embodiment, a chemotherapeutic agent is radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

Epitope: The site on an antigen recognized by an antibody as determined by the specificity of the amino acid sequence. Two antibodies are said to bind to the same epitope if each competitively inhibits (blocks) binding of the other to the antigen as measured in a competitive binding assay (see, e.g., Junghans et al., Cancer Res. 50:1495-1502, 1990). Alternatively, two antibodies have the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

Graft-Versus-Host Disease (GVHD): A common and serious complication of bone marrow or other tissue transplantation wherein there is a reaction of donated immunologically competent lymphocytes against a transplant recipient's own tissue. GVHD is a possible complication of any transplant that uses or contains stem cells from either a related or an unrelated donor.

There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines.

Immune-Mediated Disorder: A disorder in which the immune response plays a key role in the development or progression of the disease. Immune-mediated disorders include autoimmune disorders, allograft rejection, graft versus host disease and inflammatory and allergic conditions.

Immune response: A response of a cell of the immune system, such as a B cell, or a T cell, or innate immune cell to a stimulus. In one embodiment, the response is specific for a particular antigen (an “antigen-specific response”).

A “parameter of an immune response” is any particular measurable aspect of an immune response, including, but not limited to, cytokine secretion (IL-6, IL-10, IFN-γ, etc.), chemokine secretion, altered migration or cell accumulation, immunoglobulin production, dendritic cell maturation, regulatory activity, number of regulatory B cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of ³H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≦0.05 is significant, and indicates that the chance that an increase or decrease in any observed parameter is due to random variation is less than 5%. One of skill in the art can readily identify other statistical assays of use.

Immunoglobulin: A protein including one or more polypeptides substantially encoded by immunoglobulin genes. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length. Full-length immunoglobulin heavy chains are generally about 50 Kd or 446 amino acid in length. Light chains are encoded by a variable region gene at the NH2-terminus (about 110 amino acids in length) and a kappa or lambda constant region gene at the COOH—terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al., Eur. J. Immunol. 17:105, 1987; Huston et al., Proc. Natl. Acad. Sci. U.S.A., 85:5879-5883, 1988; Bird et al., Science 242:423-426, 1988; Hood et al., Immunology, Benjamin, N.Y., 2nd ed., 1984; Hunkapiller and Hood, Nature 323:15-16, 1986).

An immunoglobulin light or heavy chain variable region includes a framework region interrupted by three hypervariable regions, also called complementarity determining regions (CDR's) (see, Sequences of Proteins of Immunological Interest, E. Kabat et al., U.S. Department of Health and Human Services, 1983). As noted above, the CDRs are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715, which is herein incorporated by reference.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Exemplary conservative substitutions are those such as gly, ala; val, ile, leu; asp, glu; asn, gln; ser, thr; lys, arg; and phe, tyr (see U.S. Pat. No. 5,585,089, which is incorporated herein by reference). Humanized immunoglobulins can be constructed by means of genetic engineering, e.g., see U.S. Pat. No. 5,225,539 and U.S. Pat. No. 5,585,089, which are herein incorporated by reference.

A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., Dower et al., PCT Publication No. WO91/17271; McCafferty et al., PCT Publication No. WO92/001047; and Winter, PCT Publication No. WO92/20791, which are herein incorporated by reference), or selected from a human combinatorial monoclonal antibody library (see the Morphosys website). Human antibodies can also be prepared by using transgenic animals carrying a human immunoglobulin gene (e.g., see Lonberg et al., PCT Publication No. WO93/12227; and Kucherlapati, PCT Publication No. WO91/10741, which are herein incorporated by reference).

Interleukin (IL)-10: IL-10 is a homodimeric protein with subunits having a length of 160 amino acids. Human IL10 shows 73% amino acid homology with murine IL-10. In humans IL-10 is produced by activated CD8+ peripheral blood T-cells, by T-helper CD4(+) T-cell clones (resembling Th0, Th1, and Th2) after both antigen-specific and polyclonal activation, by B-cell lymphomas, and by monocytes following cell activation. The synthesis of IL-10 by monocytes is inhibited by IL-4 and IL-10. In the human system, IL-10 is produced by a number of cell types, and downregulates the function of Teffector cells as well as antigen presenting cells of the innate immune system. IL-10 inhibits the synthesis of a number of cytokines such as IFN-γ, IL-2 and TNF-beta in Th1 T-helper subpopulations of T-cells but not of Th2 T-helper cells.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. Similarly, an “isolated” cell has been substantially separated, produced apart from, or purified away from other cells of the organism in which the cell naturally occurs. Isolated cells can be, for example, at least 99%, at least 98%, at least 95%, at least 90%, at least 85%, or at least 80% pure. An isolated CD19⁺TIM-1⁺ B cell is separated from other types of cells, such as CD19⁺TIM-1⁻ B cells. In one embodiment, an isolated population of CD19⁺TIM-1⁺ B cells does not include a substantial amount of CD19⁺TIM-1⁻CD1d^{high}CD5⁺ B cells. Thus, in some examples, an isolated population of regulatory B cells can include less than 5%, less than 2%, less than 1% or less than 0.05% of TIM-1⁻ B cells. In some examples, CD19+TIM-1+ cells are 90%, 91%, 92%, 93%, 94% 95%. 97%, 98%, or even 99% pure. Thus, in some examples, an isolated population of regulatory B cells can include less than 10%, less than 9%, less than 8%, less than 7%, less than 6%, less than 5%, less than 4%, less than 3%, less than 2%, less than 1% or less than 0.05% of TIM-1⁻ B cells.

Leukocyte: Cells in the blood, also termed “white cells,” that are involved in defending the body against infective organisms and foreign substances. Leukocytes are produced in the bone marrow. There are 7 main types of white blood cells, subdivided between 2 main groups: polymorphonuclear leukocytes (neutrophils, eosinophils, basophils) and mononuclear leukocytes (monocytes dendritic cells, natural killer cells, and lymphocytes). Lymphocytes are comprised of B cells and T cells and make up the adaptive immune system which exhibit antigen-specific responses. Non-lymphocytes make up the innate immune system.

Mammal: This term includes both human and non-human mammals. Similarly, the term “subject” includes both human and veterinary subjects.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “fragment” refers to a portion of a polypeptide that is at least 8, 10, 15, 20 or 25 amino acids in length. The term “functional fragments of a polypeptide” refers to all fragments of a polypeptide that retain an activity of the polypeptide (e.g., the binding of an antigen). Biologically functional fragments, for example, can vary in size from a polypeptide fragment as small as an epitope capable of binding an antibody molecule to a large polypeptide capable of participating in the characteristic induction or programming of phenotypic changes within a cell. The term “soluble” refers to a form of a polypeptide that is not inserted into a cell membrane.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Pharmaceutical agent or drug: A chemical compound or composition capable of inducing a desired therapeutic or prophylactic effect when properly administered to a subject. Pharmaceutical agents include, but are not limited to, chemotherapeutic agents and anti-infective agents.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing or treating a disease: “Preventing” a disease refers to inhibiting the full development of a disease, for example in a person who is known to have a predisposition to a disease such as an immune-mediated disorder, such as an autoimmune disease. An example of a person with a known predisposition is someone with a history of an autoimmune disorder in the family, or who has been exposed to factors that predispose the subject to a condition. “Treatment” refers to a therapeutic intervention that ameliorates a sign or symptom of a disease or pathological condition after it has begun to develop.

Purified: The term purified does not require absolute purity; rather, it is intended as a relative term. Thus, for example, a purified peptide preparation is one in which the peptide or protein is more enriched than the peptide or protein is in its natural environment within a cell. Preferably, a preparation is purified such that the protein or peptide represents at least 50% of the total peptide or protein content of the preparation. In another example, a purified cell preparation is one in which the cell type of interest is significantly more enriched than the cell is in its natural environment within a cell. Preferably, a preparation is purified such that the cell represents at least 50% of the total cell content of the preparation.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded by a recombinant nucleic acid molecule.

Symptom and sign: Any subjective evidence of disease or of a subject's condition, i.e., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to any measurable parameters such as tests for immunological status or the presence of lesions in a subject.

T Cell: A white blood cell critical to the immune response. T cells include, but are not limited to, CD4⁺ T cells and CD8⁺ T cells. A CD4⁺ T lymphocyte is an immune cell that carries a marker on its surface known as cluster of differentiation 4 (CD4). These cells, classically known as helper T cells (Th cells), help orchestrate the immune response by other white blood cells, including antibody responses as well as killer T cell responses. CD8⁺ T cells carry the cluster of differentiation 8 (CD8) marker. In one embodiment, CD8 T cells are cytotoxic T lymphocytes (Tc cells) which are capable of lysing target cells by direct cell contact. These cells play a role in the elimination of virus-infected cells and tumor cells, and are involved in transplant rejection processes. In another embodiment, a CD8 cell is a suppressor T cell. Mature T cells express CD3. Regulatory T cells (Tregs) suppress immune responses of other cells. In one example, a regulatory T cell is CD4+CD25+. In additional examples, a regulatory T cells generally express CD4 and FOXP3.

T cell immunoglobulin mucin-1 (TIM-1): A member of the gene family of T cell immunoglobulin (Ig) domain and mucin domain (TIM) proteins, which are expressed in T cells, plays an important role in regulating T effector differentiation and function including that mediated by Th1, Th2, Th17, and Treg cells. The TIM gene family is located on human chromosome 5q31-33. In humans, the TIM gene family has three members, TIM-1, TIM-3, and TIM-4. (The TIM-2 gene is found only in mice). The encoded product of all members of the family is a type I membrane protein characterized by identical structural motifs including a signal peptide, Ig domain, mucin domain, transmembrane region, and intercellular tail with phosphorylation sites. TIM-1 is also referred to as KIM1 or HAVCR-1 as it was initially cloned as kidney injury molecule 1 and hepatitis A virus cellular receptor. TIM-1 is expressed on activated T cells, and upon CD4+ T cell polarization is expressed at higher levels on Th2 cells than on Th1 cells. It has been proposed that TIM-1 expressed on these T cells is a positive costimulatory molecules that can enhance cell proliferation and cytokine production. TIM-4, which is expressed on antigen presenting cells, is the natural ligand for TIM-1 (see Ueno et al., J. Clin. Invest. 118: 742-751, 2008). Administration of anti-TIM-1 antibody can increase graft survival in transplant recipients. An exemplary protein sequence for TIM-1 is provided in GENBANK® NM_{—}001099414, Jul. 4, 2010, which is incorporated herein by reference.

Therapeutically effective dose: A dose sufficient to prevent, treat, or to cause regression of the disease, or which is capable of relieving symptoms caused by the disease.

Tumor: An abnormal growth of cells, which can be benign or malignant. Cancer is a malignant tumor, which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma).

Tumor antigens (TAs): A antigen expressed on a tumor which can stimulate tumor-specific T-cell-defined immune responses. Exemplary TAs include, but are not limited to, RAGE-1, tyrosinase, MAGE-1, MAGE-2, NY-ESO-1, Melan-A/MART-1, glycoprotein (gp) 75, gp100, beta-catenin, PRAME, MUM-1, WT-1, CEA, and PR-1. Additional TAs are known in the art (for example see Novellino et al., Cancer Immunol. Immunother. 2004 Aug. 7 [Epub ahead of print]) and includes TAAs not yet identified.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Regulatory B Cells (Breg) and Their Production

Isolated regulatory B cells (Breg), and isolated populations of regulatory B cells are disclosed herein. These regulatory B cells are mammalian B cells that express T cell immunoglobulin mucin (TIM)-1 (TIM-1⁺). In some embodiments, the cells also express CD19, and are TIM-1⁺CD19⁺. In additional embodiments, these regulatory B cells also can express CD1d and CD5 (CD1d^{high}CD5⁺). In additional embodiments, the regulatory B cells express no or low levels of CD1d or do not express CD5. In some embodiments, the cells are primate cells, such as human or non-human primate cells.

In some embodiments, the regulatory B cells express interleukin-10. The ability of the cells to produce IL10 can be assessed by measuring IL-10 production in naive cells and in cultured cells stimulated with LPS (lipopolysaccharide), PMA (phorbol 12-myristate 13-acetate), ionomycin, CpG or comparable stimulatory Toll-like receptor agonists, or with an agonist of CD40 (e.g., using an antibody to CD40). Production of IL-10 by the cells can be assessed by assaying for IL-10 in the cell culture supernatant. In addition, production of IL 10 can be verified directly by intracellular cytokine staining or by Enzyme-linked immunosorbent spot (ELISPOT). Standard immunoassays known in the art can be used for such purpose (see PCT Publication No. 20091131712, which is incorporated herein by reference.

An isolated regulatory B cell population can include at least 90%, at least 91%, at least 92%, at least 93%, at least 94%, at least 95% at least 99%, or 100% regulatory B cells that express TIM-1. An isolated regulatory B cell population can include at least 90%, at least 91%, at least 92%, at least 93%, at least 94%, at least 95%, at least 99%, or 100% regulatory B cells that are TIM-1+ B cells, such as TIM-1⁺CD19⁺ B cells that produce IL-10. In some embodiments, a subset of these cells can be CD1d^{high}CD5⁺.

In some embodiments, the TIM-1⁺ B cells produce IL-10. A regulatory B cell population can include at least 90%, at least 91%, at least 92%, at least 93%, at least 94%, at least 95%, at least 99%, or 100% regulatory B cells that express TIM-1, wherein at least 5% of the TIM-1⁺ B cells produce IL-10. In some embodiments, at least 10%, at least 20%, at least 10%, at least 40%, at least 50% or at least 60% of the TIM-1⁺ B cells in the population produce IL-10. In other embodiments, a regulatory B cell population can include at least 90%, at least 91%, at least 92%, at least 93%, at least 94%, at least 95%, at least 99%, or 100% regulatory B cells that express TIM-1, and express IL-10.

Additional isolated populations of cells are disclosed herein. In some embodiments, a regulatory B cell population can include at least 90%, at least 91%, at least 92%, at least 91%, al least 94%, at least 95%, al least 99%, or 100% regulatory B cells that express TIM-1, and are CD1d^{high}CD5⁺. In other embodiments, a regulatory B cell population can include at least 90%, at least 91%, at least 92%, at least 93%, at least 94%, at least 95%, at least 99%, or 100% regulatory B cells that express TIM-1, and are CD1d^{low/l}CD5⁻.

In some embodiments, the disclosed regulatory B cells suppress resting and activated T cells. These regulatory B cells can induce generation of regulatory T cells (Tregs) from CD4⁺ T cells, for example CD4⁺FoxP3⁺ T cells, or can enhance the activity these T cells. In other embodiments, regulatory B cells may suppress other B cells, dendritic cells or macrophages. In specific non-limiting examples, the cells are human, non-human primate or murine cells.

TIM-1⁺ regulatory B cells, such as TIM-1⁺CD19⁺ B cells prevent the activation and/or expansion of other cells of the immune system. Therefore, in one embodiment, the TIM-1⁺CD19⁺ cells are immunosuppressive cells. In additional embodiments, the TIM-1⁺CD19⁺ cells are regulatory and produce IL-10.

The enriched, isolated and/or purified TIM-1⁺ regulatory B cells can be obtained from a mammalian subject, including but not limited to rodents, e.g. mice, rats; livestock, e.g. pigs, horses, cows, etc., pets, e.g. dogs, cats; and primates, e.g. humans. In one embodiment, the cells are human TIM-1+ regulatory B culls, for example, TIM-1⁺CD19⁺ regulatory B cells.

Methods are also provided herein for generating regulatory B cells. These methods include contacting a sample comprising B cells in vitro with an antibody that specifically induces TIM-1, and isolating TIM-1+ B cells, such as TIM-1⁺CD19⁺ B cells. Exemplary non-limiting methods are provided in the Examples section below.

The population of regulatory B cells can be obtained from a subject in need of therapy or suffering from a disease associated with reduced regulatory B cell activity. Thus, the cells will be autologous to the subject in need of therapy. Alternatively, the population of regulatory B cells can be obtained from a donor, preferably a histocompatibility matched donor. The regulatory B cell population can be harvested from the peripheral blood, bone marrow, spleen, or any other organ/tissue in which regulatory B cells reside in said subject or donor. The regulatory B cells can be isolated from a pool of subjects and/or donors, or from pooled blood.

When the population of regulatory B cells is obtained from a donor distinct from the subject, the donor is preferably syngeneic, but can also be allogeneic, or even xenogeneic, provided the cells obtained are subject-compatible in that they can be introduced into the subject. Allogeneic donor cells are preferably human-leukocyte-antigen (HLA)-compatible, and are typically administered in conjunction with immunosuppressive therapy. To be rendered subject-compatible, xenogeneic cells can be treated to reduce immunogenicity (Fast et al., 2004, Transfusion 44:282-5).

Methods for the isolation and quantitation of populations of cells are well known in the art, and the isolation and quantitation of regulatory B cells, such as TIM-1⁺ cells can be accomplished by any means known to one of skill in the art. Fluorescence activated cell sorting (FACS), or other cell isolation methods, can be used to sort cells that are TIM-1⁺, optionally CD19⁺, and optionally cells that express one or more of IL-10, CD1d and CD5. In one example, methods are used to isolate regulatory B cells that express TIM-1 and a pan-B cell marker (such as CD-19). The methods can isolate cells that express TIM-1 and CD19. Regulatory B cells can also be isolated that express TIM-1 and CD19 and are CD1d^{high}CD5⁺ or that are non-CD1d^{high}CD5⁺ (CD1d^{low/-}CD5⁻, CD1d^{low/-}CD5⁺, or CD1d^{high}CD5⁻) or that belong to any other B cell subpopulation. In one embodiment, labeled antibodies specifically directed to one or more cell surface markers are used to identify and quantify TIM⁺ regulatory B cells, such as TIM-1⁺CD19⁺ cells, for example TIM-1⁺CD19⁺CD1d^{high}CD5⁺ cells or TIM-1⁺CD19⁺ non-CD1d^{high}CD5⁺ cells. The antibodies can be conjugated to other compounds including, but not limited to, enzymes, magnetic beads, colloidal magnetic beads, haptens, fluorochromes, metal compounds, radioactive compounds or drugs. The enzymes that can be conjugated to the antibodies include, but are not limited to, alkaline phosphatase, peroxidase, urease and B-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For some additional fluorochromes that can be conjugated to antibodies see Haugland, R. P., Handbook of Fluorescent Probes and Research Products, published by Molecular Probes, 9^{th} Edition (2002), although many fluorochomes are known in the art and are commercially available. The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include, but are not limited to, technetium 99 (⁹⁹Tc), ¹²⁵I, and amino acids comprising any radionuclides, including, but not limited to, ¹⁴C, ³H and ³⁵S.

In some examples, TIM-1⁺ cells, such as TIM-1⁺CD19⁺ cells, are isolated by contacting the cells with an appropriately labeled antibody to identify the cells of interest followed by a separation technique such as FACs or antibody-binding beads. However, other techniques of differing efficacy may be employed to purify and isolate desired populations of cells. The separation techniques employed should maximize the retention of viability of the fraction of the cells to be collected. The particular technique employed will, of course, depend upon the efficiency of separation, cytotoxicity of the method, the ease and speed of separation, and what equipment and/or technical skill is required.

Additional separation procedures may include magnetic separation, using antibody-coated magnetic beads, affinity chromatography, cytotoxic agents, either joined to a monoclonal antibody or used in conjunction with complement, and “panning,” which utilizes a monoclonal antibody attached to a solid matrix, or another convenient technique. Antibodies attached to magnetic beads and other solid matrices, such as agarose beads, polystyrene beads, hollow fiber membranes and plastic Petri dishes, allow for direct separation. Cells that are bound by the antibody can be removed from the cell suspension by simply physically separating the solid support from the cell suspension. The exact conditions and duration of incubation of the cells with the solid phase-linked antibodies will depend upon several factors specific to the system employed. The selection of appropriate conditions, however, is well known in the art.

Unbound cells then can be eluted or washed away with physiologic buffer after sufficient time has been allowed for the cells expressing a marker of interest (for example, TIM or CD19⁺) to bind to the solid-phase linked antibodies. The bound cells are then separated from the solid phase by any appropriate method, depending mainly upon the nature of the solid phase and the antibody employed, and quantified using methods well known in the art. In one specific, non-limiting example, bound cells separated from the solid phase are quantified by flow cytometry.

Antibodies may be conjugated to biotin, which then can be removed with avidin or streptavidin bound to a support, or fluorochromes, which can be used with FACS to enable cell separation and quantitation, as known in the art.

In another embodiment, an apheresis procedure employing an automated apheresis instrument (such as the CS-3000 blood cell separator, Baxter Health Care, Deerfield, Ill., or equivalent machine) can be used to collect cells from a subject. In a specific, non-limiting example, labeled antibodies specifically directed to one or more cell surface markers are used to identify and quantify the TIM-1⁺ CD19⁺ cells, as described above.

Regulatory B cells, such as TIM-1⁺ B cells, can also be isolated by negatively selecting against cells that are not regulatory B cells. This can be accomplished by performing a lineage depletion, wherein cells are labeled with antibodies for particular lineages such as the T lineage, the macrophage/monocyte lineage, the dendritic cell lineage, the granulocyte lineages, the erythrocytes lineages, the megakaryocytes lineages, and the like. Cells labeled with one or more lineage specific antibodies can then be removed either by affinity column processing (where the lineage marker positive cells are retained on the column), by affinity magnetic beads or particles (where the lineage marker positive cells are attracted to the separating magnet), by “panning” (where the lineage marker positive cells remain attached to the secondary antibody coated surface), or by complement-mediated lysis (where the lineage marker positive cells are lysed in the presence of complement by virtue of the antibodies bound to their cell surface). Another lineage depletion strategy involves tetrameric complex formation. Cells are isolated using tetrameric anti-human antibody complexes (for example, complexes specific for multiple markers on multiple cell types that are not markers of regulatory B cells, and magnetic colloid in conjunction with STEMSTEP™ columns (Stem Cell Technologies, Vancouver, Canada). The cells can then optionally be subjected to centrifugation to separate cells having tetrameric complexes bound thereto from all other cells.

In a certain embodiment, the enriched/purified population of regulatory B cells can be stored for a future use. In this regard, the regulatory B cell population can be cryopreserved. Cryopreservation is a process where cells or whole tissues are preserved by cooling to low sub-zero temperatures, such as 77 K or −196° C. in the presence of a cryoprotectant. Storage by cryopreservation includes, but is not limited to, storage in liquid nitrogen, storage in freezers maintained at a constant temperature of about 0° C., storage in freezers maintained at a constant temperature of about −20° C., storage in freezers maintained at a constant temperature of about −80° C., and storage in freezers maintained at a constant temperature of lower than about −80° C. In one aspect of this embodiment, the cells may be “flash-frozen,” such as by using in ethanol/dry ice or in liquid nitrogen prior to storage. In another aspect of this embodiment, the cells can be preserved in medium comprising a cryprotectant including, but not limited to dimethyl sulfoxide (DMSO), glycerol, ethylene glycol, propylene glycol, sucrose, and trehalose. Other methods of storing biological matter are well known to those of skill in the art, see for example U.S. Patent application publication No. 2007/0078113, incorporated by reference herein.

In another embodiment, the isolated regulatory B cells are expanded to increase the number of cells. Expansion of the regulatory B cell population can be achieved by contacting the population of regulatory B cells with stimulatory composition sufficient to cause an increase in the number of regulatory B cells. This may be accomplished by contacting the isolated TIM-1⁺ B cells with a mitogen, cytokine, growth factor, or antibody, such as an antibody that specifically binds to the B cell receptor or to TIM-1. This may also be accomplished by contacting the isolated TIM-1+ B cells with a fusion protein or ligand that specifically binds to TIM-1. The regulatory B cells can be expanded at least 10-fold, such as at least 50, 100, 200, 300, 500, 800, 1000, 10,000, or 100.000-fold. Generally, the expanded regulatory B cell population retains all of the genotypic, phenotypic, and functional characteristics of the original population. The isolated regulatory B cells can also be expanded by treating the cells with an antibody that activates or induces the expression of TIM-1, treating the cells with a growth factor, treating the cells with a mitogen, treating the cells with antibodies that specifically bind the B cell receptor, treating the cells with antibodies that specifically bind CD40, treating the cells with antibodies that specifically bind IgM, or a combination thereof. In another embodiment, B cells may be expanded first and TIM-1+ B cells isolated after expansion and TIM-1 induction.

### Method for Inducing Immunosuppression in a Subject

A method is also disclosed herein for inducing immunosuppression in a subject, such as for treating or preventing an immune-mediated disorder. The method includes administering to the subject a therapeutically effective amount of TIM-1⁺ regulatory B cells, such as TIM-1⁺CD19⁺ regulatory B cells (for example, TIM-1⁺CD19⁺ cells, TIM-1+CD19+CD1d^{high}CD5⁺ cells, TIM-1+IgM⁺IgD⁻CD21^{hi}CD23⁻ cells, or TIM-1⁺CD19⁺ non-CD1d^{high}CD5+⁻ cells, such as CD1d^{low}CD5⁺, CD1d^{low}CD5⁻ cells, CD1d^{high}CD5⁻ cells, thereby treating or preventing the immune-mediated disorder in the subject.

In some embodiments the TIM-1⁺CD19⁺ cells express IL-10. In some embodiments, the regulatory B cells are autologous or syngeneic. However the cells can be allogeneic or xenogeneic. In some embodiments, the regulatory B cells are isolated from the patient themself, so that the cells are autologous. If the regulatory B cells are allogeneic or xenogeneic, the regulatory B cells can be pooled from several donors. The cells are administered to the subject of interest in an amount sufficient to control, reduce, or eliminate symptoms and signs of the disease being treated.

In some embodiments, the regulatory B cell is contacted with an antigen specific to a disorder, such as an autoimmune disorder, prior to introducing them to a subject. For example, the regulatory B cells may be exposed to an autoantigen such as insulin or GAD-65 prior to administration to a subject to prevent or treat diabetes.

In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment the subject is the recipient of a transplanted organ and TIM-1+ regulatory B cells are used to prevent and/or treat rejection. Any of the populations of regulatory B cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Regulatory B cells, such as immunosuppressive TIM-1⁺CD19⁺ cells, can be administered prior to transplantation, concurrently with transplantation, or following transplantation. In some embodiments, the regulatory B cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of regulatory B cells occurs 3-5 days prior to transplantation.

In other embodiments, the subject has or is at risk of developing graft versus host disease. The subject can be the recipient of a bone marrow transplant.

A regulatory B cell subset administered to a patient that is receiving a transplant can be sensitized with antigens specific to the transplanted material prior to administration. According to this embodiment, the transplant recipient will have a decreased immune/inflammatory response to the transplanted material and, as such, the likelihood of rejection of the transplanted tissue is minimized. Similarly, with regard to the treatment of graft versus host disease, the regulatory B cell subset can be sensitized with antigens specific to the host. According to this embodiment, the reciptient will have a decreased immune/inflammatory response to self antigens.

In a further embodiment, administration of a therapeutically effective amount of regulatory B cells to a subject treats or inhibits inflammation in the subject. Thus, the method includes administering a therapeutically effective amount of regulatory B cells to the subject to inhibit the inflammatory process. Examples of inflammatory disorders include, but are not limited to, asthma, encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacteria infections. The methods disclosed herein can also be used to treat allergic disorders.

Administration of regulatory B cells can be utilized whenever immunosuppression or inhibition of inflammation is desired, for example, at the first sign or symptoms of a disease or inflammation. These may be general, such as pain, edema, elevated temperature, or may be specific signs or symptoms related to dysfunction of affected organ(s). For example in renal transplant rejection there may be an elevated serum creatinine level, whereas in GVHD, there may be a rash, and in asthma, there may be shortness of breath and wheezing.

Administration of regulatory B cells can also be utilized to prevent immune-mediated disease in a subject of interest. For example, regulatory B cells can be administered to a subject that will be a transplant recipient prior to the transplantation. In another example, regulatory T cells are administered to a subject receiving allogeneic bone marrow transplants without T cell depletion. In a further example, regulatory B cells can be administered to a subject with a family history of diabetes. In other example, regulatory B cells are administered to a subject with asthma in order to prevent an asthma attack. In some embodiments, a therapeutically effective amount of regulatory B cells is administered to the subject in advance of a symptom. The administration of the regulatory B cells results in decreased incidence or severity of subsequent immunological event or symptom (such as an asthma attack), or improved patient survival, compared to patients who received other therapy not including regulatory B cells.

In a specific, non-limiting example, the regulatory B cells are TIM-1⁺ cells, such as TIM-1⁺CD19⁺ cells, such as TIM-1+CD19+ regulatory B cells that produce IL-10, for example TIM-1⁺CD19⁺CD1d^{high}CD5⁺ B cells, TIM-1+IgM⁺IgD⁻ CD21^{hi}CD23⁻ cells, or TIM-1⁺CD19⁺ non-CD1d^{high}CD5+⁻ cells, such as CD1d^{low}CD5⁺, CD1d^{low}CD5⁻ cells, CD1d^{high}CD5⁻ cells. In some embodiments, the population of cells administered to a subject are, for example, about 95%, about 96%, about 97%, about 98%, about 99% or 100% pure.

The effectiveness of treatment can be measured by many methods known to those of skill in the art. In one embodiment, a white blood cell count (WBC) is used to determine the responsiveness of a subject's immune system. A WBC measures the number of white blood cells in a subject. Using methods well known in the art, the white blood cells in a subject's blood sample are separated from other blood cells and counted. Normal values of white blood cells are about 4,500 to about 10,000 white blood cells/μl. Lower numbers of white blood cells can be indicative of a state of immunosuppression in the subject.

In another embodiment, immunosuppression in a subject may be determined using a T-lymphocyte count. Using methods well known in the art, the white blood cells in a subject's blood sample are separated from other blood cells. T-lymphocytes are differentiated from other white blood cells using standard methods in the art, such as, for example, immunofluorescence or FACS. Reduced numbers of T-cells, or a specific population of T-cells can be used as a measurement of immunosuppression. A reduction in the number of T-cells, or in a specific population of T-cells, compared to the number of T-cells (or the number of cells in the specific population) prior to treatment can be used to indicate that immunosuppression has been induced.

In additional embodiments, tests to measure T cell activation, proliferation, or cytokine responses including those to specific antigens are performed. In some examples, the number of Treg or Breg cells can be measured in a sample from a subject. In additional examples, cytokines are measured in a sample, from a subject, such as IL-10.

In other examples, to assess inflammation, neutrophil infiltration at the site of inflammation can be measured. In order to assess neutrophil infiltration myeloperoxidase activity can be measured. Myeloperoxidase is a hemoprotein present in azurophilic granules of polymorphonuclear leukocytes and monocytes. It catalyzes the oxidation of halide ions to their respective hypohalous acids, which are used for microbial killing by phagocytic cells. Thus, a decrease in myeloperoxidase activity in a tissue reflects decreased neutrophil infiltration, and can serve as a measure of inhibition of inflammation.

In another example, effective treatment of a subject can be assayed by measuring cytokine levels in the subject. Cytokine levels in body fluids or cell samples are determined by conventional methods. For example, an immunospot assay, such as the enzyme-linked immunospot or “ELISPOT” assay, can be used. The immunospot assay is a highly sensitive and quantitative assay for detecting cytokine secretion at the single cell level. Immunospot methods and applications are well known in the art and are described, for example, in Czerkinsky et al., J. Immunol. Methods 110:29-36, 1988; Olsson et al. J. Clin. Invest. 86:981-985, 1990; and EP 957359. Variations of the standard immunospot assay are well known in the art and can be used to detect alterations in cytokine production in the methods of the disclosure (see, for example, U.S. Pat. No. 5,939,281 and U.S. Pat. No. 6,218,132).

Antibodies suitable for use in immunospot assays, which are specific for secreted cytokines, as well as detection reagents and automated detection systems, are well known in the art and generally are commercially available. Appropriate detection reagents are also well known in the art and commercially available, and include, for example, secondary antibodies conjugated to fluorochromes, colored beads, and enzymes whose substrates can be converted to colored products (for example, horseradish peroxidase and alkaline phosphatase). Other suitable detection reagents include secondary agents conjugated to ligands (for example, biotin) that can be detected with a tertiary reagent (for example, streptavidin) that is detectably labeled as above.

Other methods for measuring cytokine levels in the subject are well known in the art, and can be used as an alternative to immunospot assays. Such methods include ELISA, which can be used to measure the amount of cytokine secreted by T-cells into a supernatant (see, for example, Vandenbark et al., Nature Med. 2:1109-1115, 1996). Alternatively, the expression of cytokine mRNA can be determined by standard immunological methods, which include reverse transcriptase polymerase chain reaction (RT-PCR) and in-situ hybridization.

In another embodiment, administration of a therapeutically effective amount of TIM-1⁺ regulatory B cells (such as TIM-1⁺CD19⁺ cells) to a subject induces the production or activity of regulatory T cells, such as CD4⁺CD25⁺ of CD4+Foxp3+ suppressive T cells. In further embodiments, administration of a therapeutically effective amount of TIM-1⁺ regulatory B cells (such as TIM-1⁺CD19⁺ cells) decreases the proliferation of CD4⁺ and/or CD8⁺ T cells. In further embodiments, administration of a therapeutically effective amount of TIM-1⁺ regulatory B cells (such as TIM-1⁺CD19⁺ cells) reduces production of antibodies produced by the subject's non-regulatory B cells, that are involved in the immune-mediated disease. In further embodiments, regulatory B cells may inhibit influx of inflammatory cells or damage mediated by innate immune cells. Thus, all of these cell types can be measured. In a further embodiment, cytokine production can be measured.

Suppression of proliferation can be evaluated using many methods well known in the art. In one embodiment, cell proliferation is quantified by measuring [³H]-thymidine incorporation. Proliferating cells incorporate the labeled DNA precursor into newly synthesized DNA, such that the amount of incorporation, measured by liquid scintillation counting, is a relative measure of cellular proliferation. In another embodiment, cell proliferation is quantified using the thymidine analogue 5-bromo-2′-deoxyuridine (BrdU) in a proliferation assay. BrdU is incorporated into cellular DNA in a manner similar to thymidine, and is quantified using anti-BrdU mAbs by flow cytometry.

In a further embodiment, cell proliferation may be determined based upon the reduction of the tetrazolium salt 3,[4,5-dimethylthiazol-2-yl]-2,5-diphenyltetrazolium bromide (MTT). The tetrazolium ring of MTT is reduced to formazan, which is blue in color, by the succinate-tetrazolium reductase system active only in viable cells. The intensity of the resulting color change indicates the enzymatic activity of living cells. In actively proliferating cells, MTT conversion increases, whereas in senescent and dying cells, the rate of MTT conversion slows. Comparison of this value to an untreated control provides a measure of the change in cellular proliferation. In further embodiment, proliferation is measured by dilution of the fluorescent dye, Carboxyfluorescein succinimidyl ester (CFSE). Cells of interest are stained with CFSE which is taken up and affixed to cellular proteins. As cells divide, the proteins are divided up equally amongst daughter cells, and the fluorescence intensity decreases by half with each division.

Therapeutically effective amounts of regulatory B cells can be administered by a number of routes, including parenteral administration, for example, intravenous, intraperitoneal, intramuscular, intrasternal, or intraarticular injection, or infusion.

The therapeutically effective amount of regulatory B cells for use in inducing immunosuppression or treating or inhibiting inflammation is that amount that achieves a desired effect in a subject being treated. For instance, this can be the amount of regulatory B cells necessary to inhibit advancement, or to cause regression of an autoimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The regulatory B cell population can be administered in treatment regimes consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of regulatory B cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ regulatory B cells/m². In a certain embodiment, the dose used in the treatment of human subjects ranges from about 3.8×10⁹ to about 3.8×10¹⁰ regulatory B cells/m². In additional embodiments, a therapeutically effective amount of regulatory B cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of regulatory B cells is readily determined by one of skill in the art based on the age, weight, sex, and physiological condition of the subject. Effective doses can be extrapolated from dose-response curves derived from in vitro or animal model test systems.

In an exemplary adoptive cell transfer protocol, a mixed population of B cells is initially extracted from a target donor. The regulatory B cells isolated from the donor may be isolated from any location in the donor in which they reside including, but not limited to, the blood, spleen, lymph nodes, and/or bone marrow of the donor. Depending on the application, the B cells may be extracted from a healthy donor; a subject who has a disease that is in a period of remission or during active disease; or from the organs, blood, or tissues of a cadaveric donor. In the case of the latter, the donor is an organ donor. In yet another embodiment, the regulatory B cells can be obtained from the subject of interest, expanded or activated and returned to the subject.

Harvested TIM-1+ B cell lymphocytes may be separated by flow cytometry or other cell separation techniques, described herein, and then transfused to a recipient. Alternatively, the cells may be stored for future use. Optionally, the cells are expanded prior to use and/or treated with an antibody, fusion protein, or ligand that specifically binds TIM-1 or augments TIM-1 expression.

B regulatory cells can be identified using TIM-1 (as above) and reinfused into subjects directly, or after in vitro activation or stimulation with antigen, mitogen, anti-B cell receptor antibody, or additional stimulatory agents (e.g. anti-CD40, LPS) alone or in combination to augment B regulatory cell activity. In another embodiment, the number of B regulatory cells might be expanded in vitro (using mitogen, antigen, or stimulatory agents such as those listed above) to increase their cell number. In this embodiment, regulatory cells within the B cell population may be identified using TIM-1 before in vitro expansion, after expansion, or both. Anti-TIM-1 antibodies, fusion proteins, or ligands, may be used to further stimulate TIM-1 or IL-10 expression in B cells in vitro. Enrichment for regulatory B cells is critical to avoid infusing potential inflammatory B cells back into the subject.

The isolated regulatory TIM-1⁺ B cells, such as TIM-1⁺CD19⁺ CD1d^{high}CD5⁺ B cells, or TIM-1⁺CD19+ B cells that secrete IL-10, or any of the regulatory B cell populations disclosed herein, can be administered in a pharmaceutically acceptable carrier, such as buffered saline or another medium suitable for administration to a subject. The regulatory B cells can be administered in conjunction with other cells, such as regulatory T cells.

The therapeutically effective amount of regulatory B cells can be administered with another agent. In one embodiment, the agent is an antibody that specifically binds TIM-1 or a functional fragment thereof. The antibody can be a monoclonal antibody, such as a humanized antibody, or fully human antibody. In one embodiment, compositions containing isolated populations of regulatory B cells can also contain one or more additional pharmaceutical agents, such as a monoclonal antibody that specifically binds TIM-1 or a functional fragment thereof, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In a similar embodiment, TIM-1 may be targeted using a fusion protein or a natural ligand. In addition, immunosuppressive or tolerogenic agents including but not limited to calcineurin inhibitors (e.g. cyclosporin and tacrolimus); mTOR inhibitors (e.g. Rapamycin); mycophenolate mofetil, antibodies (e.g. recognizing CD3, CD4, CD40, CD154, CD45, IVIG, or B cells); chemotherapeutic agents (e.g. Methotrexate, Treosulfan, Busulfan); irradiation; or chemokines, interleukins or their inhibitors (e.g. BAFF, IL-2, anti-IL-2R, IL-4, JAK kinase inhibitors) can be administered.

Such additional pharmaceutical agents can be administered before, during, or after administration of the regulatory B cells, depending on the desired effect. This administration of the cells and the agent can be by the same route or by different routes, and either at the same site or at a different site.

In another aspect of this embodiment, the regulatory B cells obtained from the donor can be introduced into a recipient at a desired location, so as to specifically target the therapeutic effects of the regulatory B cell population. Such techniques can be accomplished using implantable immune modulation devices, such as virtual lymph nodes, see U.S. Patent Application Publication No. 2003/0118630; PCT Publication No. WO 1999/044583; and U.S. Pat. No. 6,645,500, which all are incorporated by reference herein. According to this embodiment, a regulatory B cell population is isolated from a donor as described above, added to an implantable immune modulation device, and the device then can be implanted into a recipient at a location where the therapeutic effects of the regulatory B cell population are needed.

### Methods for Treating Tumors

Methods are provided herein for inducing an immune response to a tumor antigen in a subject with a tumor. These methods include decreasing the number or activity of TIM-1⁺ regulatory B cells (such as TIM-1⁺CD19⁺ regulatory B cells) in the subject; with or without administering the tumor antigen or a nucleic acid encoding the tumor antigen to the subject, thereby inducing an immune response to the tumor antigen in the subject. Regulatory B cells can be inhibited or killed by delivering toxins or siRNA/miRNA utilizing cell surface receptors expressed by the cells. In some embodiments, reducing the number or function of TIM-1⁺ regulatory B cells in the subject comprises administering to the subject a therapeutically effective amount of an antibody that specifically binds TIM-1 and depletes these cells or an antibody that specifically inhibit Breg function.

Specifically, the monoclonal antibody RMT1-10 promotes Breg, inhibits the immune response promotes Treg and induces Th2 cytokines, while anti-TIM-1 mAb 3B3 augments inflammatory/immune disorders, augments Th1 and Th17 responses, and inhibits Tregs. Low affinity anti-TIM-1 mAbs can inhibit the immune response through augmenting Th2 cytokines, and promoting Breg number and function. In contrast, high affinity anti-TIM-1 mAbs augment the immune response by promoting Th1 and Th17 cytokines and inhibiting Treg number. Thus, in another embodiment, antibodies or fusion proteins binding TIM-1 can be used to reduce Breg function. In a further embodiment, anti-TIM-1 mAbs can be used to reduce Breg number by inhibiting conversion from TIM-1− B cells to TIM-1+ B cells.

The method can also include administering to the subject a therapeutically effective amount of an additional chemotherapeutic agent. The antibody that targets the regulatory B cell subset, such as an antibody that specifically binds TIM-1, can be further conjugated to a cytotoxic agent, using methods known in the art (see, e.g., DiJoseph et al., 2004, Clin. Cancer Res. 10:8620-9).

Non-limiting examples of cytotoxic agents include antimetabolites (such as cytosine arabinoside, aminopterin, methotrexate, 6-mercaptopurine, 6-thioguanine, cytarabine, and 5-fluorouracil decarbazine); alkylating agents (such as mechlorethamine, thiotepa chlorambucil, melphalan, carmustine (BCNU) and lomustine (CCNU), cyclophosphamide, busulfan, dibromomannitol, streptozotocin, mitomycin C, cis-dichlorodiammine-platinum (II) (CDDP), and cisplatin); vinca alkaloid; anthracyclines (such as daunorubicin and doxorubicin); antibiotics (such as dactinomycin, bleomycin, mithramycin, and anthramycin (AMC)); calicheamicin; CC-1065 and derivatives thereof; auristatin molecules (such as auristatin PHE, bryostatin-1, and dolastatin-10; see Woyke et al., Antimicrob. Agents Chemother 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad, et al., Int. J. Oncol. 15:367-72 (1999)); DNA-repair enzyme inhibitors (such as etoposide or topotecan); kinase inhibitors (such as compound ST 1571, imatinib mesylate (Kantarjian et al., Clin. Cancer Res. 8(7):2167-76 (2002)); demecolcine; and other cytotoxic agents (such as paclitaxel, cytochalasin B, gramicidin D, ethidium bromide, emetine, mitomycin, etoposide, tenoposide, vincristine, vinblastine, colchicine, doxorubicin, daunorubicin, dihydroxy anthracenedione, mitoxantrone, mithramycin, actinomycin D, 1-dehydrotestosterone, glucorticoids, procaine, tetracaine, lidocaine, propranolol, and puromycin and analogs or homologues thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459; farnesyl transferase inhibitors (for example, R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305; topoisomerase inhibitors (such as camptothecin, irinotecan, SN-38, topotecan, 9-aminocamptothecin, GG211 (GI 147211), DX-8951f, IST-622, rubitecan, pyrazoloacridine, XR5000, saintopin, UCE6, UCE1022, TAN-1518A, TAN 1518B, KT6006, KT6528, ED-110, NB-506, ED-110, NB-506, and rebeccamycin); bulgarein; DNA minor groove binders such as Hoechst dye 33342 and Hoechst dye 33258; nitidine; fagaronine; epiberberine; coralyne; beta-lapachone; BC-4-1; antisense oligonucleotides (such as those disclosed in the U.S. Pat. Nos. 6,277,832, 5,998,596, 5,885,834, 5,734,033, and 5,618,709); adenosine deaminase inhibitors (e.g., fludarabine phosphate and 2-chlorodeoxyadenosine); and pharmaceutically acceptable salts, solvates, clathrates, and prodrugs thereof.

In another embodiment, the antibody that targets the regulatory B cell population can be conjugated to a radioactive metal ion, such as the alpha-emitters ²¹′astatine, ²¹²bismuth, ²¹³bismuth; the beta-emitters ¹³′iodine, ⁹⁰yttrium, ¹⁷⁷lutetium, ¹⁵³samarium, and ¹⁰⁹palladium; or macrocyclic chelators useful for conjugating radiometal ions, including but not limited to, ¹³¹indium, ¹³¹L, ¹³¹yttrium, ¹³¹holmium, ¹³¹samarium, to polypeptides or any of those listed supra. In certain embodiments, the macrocyclic chelator is 1,4,7,10-tetraazacyclododecane-N,N′,N″,N′″-tetraacetic acid (DOTA), which can be attached to the antibody via a linker molecule. Such linker molecules are commonly known in the art and described in Denardo, et al, 1998, Clin Cancer Res 4(10):2483-90; Peterson, et al, 1999, Bioconjug Chem 10(4):553-7; and Zimmerman, et al., 1999, Nucl Med Biol 26(8):943-50). In still another embodiment, the antibody that targets the regulatory B cell population is conjugated to a proteinaceous agent that modifies a given biological response and leads to cytotoxicity. In one embodiment, the antibody is conjugated to a plant-, fungus-, or bacteria-derived toxin. Non-limiting examples of such toxins include A chain toxins, ribosome inactivating proteins, ricin A, deglycosylated ricin A chain, abrin, alpha sarcin, aspergillin, restrictocin, ribonucleases, diphtheria toxin, bacterial endotoxin, saporin toxin, Granzyme B or the lipid A moiety of bacterial endotoxin, cholera toxin, or Pseudomonas exotoxin and derivatives and variants thereof.

The agents described herein can be administered either systemically or locally. A typical pharmaceutical composition for intravenous administration includes about 0.1 to 10 mg of antibody per subject per day. Dosages from 0.1 up to about 100 mg per subject per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

Antibodies may be provided in lyophilized form and rehydrated with sterile water before administration, although they are also provided in sterile solutions of known concentration. The antibody solution is then added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of RITUXAN® in 1997. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

The agent that inhibits TIM-1⁺ regulatory B cells can be administered with a tumor antigen, or a nucleic acid encoding a tumor antigen. Tumor antigens are proteins that are produced by tumor cells that elicit an immune response, particularly T-cell mediated immune responses. The tumor antigen can be any tumor antigen, which are well known in the art and include, for example, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, macrophage colony stimulating factor, prostase, prostate-specific antigen (PSA), PAP, NY-ESO-1, LAGE-1a, p53, prostein, PSMA, Her2/neu, survivin and telomerase, prostate-carcinoma tumor antigen-1, MAGE, ELF2M, neutrophil elastase, ephrinB2, CD22, insulin growth factor (IGF)-I, IGF-II, IGF-I receptor and mesothelin. A list of selected tumor antigens and their associated tumors are shown below in Table 2.

TABLE 2
Exemplary tumors and their tumor antigens

| Tumor | Tumor Associated Target Antigens |
|---|---|
| Acute myelogenous leukemia | Wilms tumor 1 (WT1), PRAME, PR1, |
| | proteinase 3, elastase, cathepsin G |
| Chronic myelogenous leukemia | WT1, PRAME, PR1, proteinase 3, |
| | elastase, cathepsin G |
| Myelodysplastic syndrome | WT1, PRAME, PR1, proteinase 3, |
| | elastase, cathepsin G |
| Acute lymphoblastic leukemia | PRAME |
| Chronic lymphocytic leukemia | Survivin |
| Non-Hodgkin's lymphoma | Survivin |
| Multiple myeloma | NY-ESO-1 |
| Malignant melanoma | MAGE, MART, Tyrosinase, |
| | PRAME GP100 |
| Breast cancer | WT1, herceptin, epithelial tumor |
| | antigen (ETA) |
| Lung cancer | WT1 |
| Ovarian cancer | CA-125 |
| Prostate cancer | PSA |
| Pancreatic cancer | CA19-9, RCAS1 |
| Colon cancer | CEA |
| Renal cell carcinoma (RCC) | Fibroblast growth factor 5 |
| Germ cell tumors | AFP |

Suitable subjects may include those diagnosed with a cancer. Any type of cancer can be treated in accordance with this method of the invention. Non-limiting examples of cancers include: leukemias, such as but not limited to, acute leukemia, acute lymphocytic leukemia, acute myelocytic leukemias, such as, myeloblastic, promyelocy e, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocyte, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al, 1985, Medicine, 2d Ed., J.B. Lippincott Co., Philadelphia and Murphy, 1997, Informed Decisions: The Complete Book of Cancer Diagnosis, Treatment, and Recovery, Viking Penguin, Penguin Books U.S.A., Inc., United States of America).

A therapeutically effective amount of the antibody will depend upon the severity of the disease and the general state of the patient's health. A therapeutically effective amount of the antibody is that which provides either subjective relief of a symptom(s) or an objectively identifiable improvement as noted by the clinician or other qualified observer. These compositions can be administered in conjunction with another chemotherapeutic agent, either simultaneously or sequentially.

Many chemotherapeutic agents are presently known in the art. In one embodiment, the chemotherapeutic agents is selected from the group consisting of mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, e.g. anti-androgens, and anti-angiogenesis agents.

### Method for Diagnosis and Monitoring

In another embodiment, methods are provided for diagnosing a subject that has a disease that is associated with altered regulatory B cell activity. In another embodiment, a subject with a predisposition to a certain disease can be identified. In a further embodiment, the effectiveness of a therapeutic regimen is assessed. The method can assess the immune status of a subject, for example to determine if the regulatory B cell activity in the subject is deficient or augmented.

The regulatory B cells can be obtained from a subject of interest, such as a subject suspected of having a particular disease or condition, a subject suspected of having a predisposition to a particular disease or condition, or a subject who is undergoing therapy for a particular disease or condition. Regulatory B cells can be collected from any location in which they reside in the subject including, but not limited to, blood, spleen, thymus, lymph nodes, and bone marrow. The isolated regulatory B cells may be analyzed directly, or they can be stored until the assay is performed, such as by freezing.

In some embodiments, the methods include selecting a subject with an immune-mediated disorder or suspected of having an immune-mediated disorder; and detecting the number of TIM-1⁺ regulatory B cells, such as CD19⁺TIM-1⁺ B cells, or the ratio of CD19+TIM-1⁺ to TIM-1⁻ B cells in a biological sample from the subject. In other embodiments, the methods include measuring the number of regulatory B cells with specificity to a certain antigen associated with a specific disease, and measuring the number of regulatory B cells with any specificity. Optionally, the methods can include measuring the expression of IL-10. In some embodiments, the methods include measuring the number of TIM-1⁺CD19⁺ CD1d^{high}CD5⁺ B cells, and/or TIM-1⁺CD19⁺CD1d^{low/-}CD5⁻ B cells.

In some embodiments, the number of TIM-1+ regulatory B cells, such as CD19+TIM-1+ B, TIM-1⁺CD19⁺CD1d^{high}CD5⁺ B cells, and/or TIM-1⁺CD19⁺ TIM-1+IgM⁺IgD⁻CD21^{hi}CD23⁻ cells, or the ratio of regulatory to non-regulatory B cells is compared to a control. The control can be the number of these regulatory B cells or ratio of regulatory to non-regulatory B cells in a sample from healthy subject or a standard value. A higher number (or ratio) of CD19⁺TIM-1⁺ regulatory B cells as compared to a control indicates the subject is immunosuppressed. A lower number of CD19⁺TIM-1⁺ regulatory B cells as compared to a control (or lower ratio of CD19⁺TIM-1+ to TIM-1− cells) indicates the subject has an immune-mediated disorder or exacerbation of such a disorder, such as inflammation, graft-versus host disease, transplant rejection, or an autoimmune disorder. Methods for the quantitation of cells are known in the art, and may include pre-labeling the sample directly or indirectly; adding a second stage antibody that binds to the antibodies or to an indirect label, for example, labeled goat anti-human serum, rat anti-mouse, and the like. For example, see U.S. Pat. No. 5,635,363. A variety of assays, including FACS and immunohistochemistry, can be utilized. Generally, assays will include various negative and positive controls, as known in the art.

Onset or predisposition to a disease, such as those identified above, can be diagnosed according to these embodiments alone or in conjunction with other diagnostic tests. In particular, predisposition to or onset of overt autoimmune and inflammatory disease, may be associated with diminished levels of regulatory B cells. In some embodiments, predisposition to or onset of diseases such as cancer can be diagnosed based on the detection of increased regulatory B cells in a subject. In another embodiment, a subject diagnosed with a given disease can be monitored for disease exacerbation, recurrence, or progression. Formats for patient sampling include time courses that follow the progression of disease, comparisons of different patients at similar disease stages, such as early onset, acute stages, recovery stages; and tracking a patient during the course of response to therapy.

In an aspect of this embodiment, the numbers (or ratio) of regulatory B cells can be monitored over the course of a given therapy as a measure response to the therapy. As a non-limiting example, TIM-1⁺ B cell number (or ratio) may be used to monitor response to therapy with any agent aiming to inhibit or decrease the immune-mediated disorder. This may include any immunosuppressive or immunomodulatory agent designed to treat or prevent an immune-mediated disorder. It may also include anti-TIM-1 or any other therapy aiming to directly augment the number or activity of regulatory B cells to treat or prevent an immune-mediated disorder. In this embodiment, an increase in TIM-1⁺ B cells (or ratio) would indicate effectiveness of the therapy.

In additional embodiments, the number of Breg cells specific for an antigen of interest are measured. An increase in the number of Breg cells specific for the antigen of interest indicates the subject is immunosuppressed. A number of methods can be used to assess the number or activity of Bregs specific for an antigen of interest. In some examples, Breg can be added to autologous T cells that are stimulated with a specific antigen or combination of relevant antigens (tumor antigen, alloantigen, etc). If the B cells do not recognize the antigen of interest, they do not supress the T cells in vitro (proliferation of the T cells or sytokine expression can be meaused). In other examples, the induction of a cytokine, such as IL-10, is assessed after exposing TIM-1+ Breg to the antigen of interest. Furthermore, the production of IL-10 or the amount of TIM-1 expression can be determined in response to a panel of relevent antigens.

Thus, the method can include detecting the number/ratio of TIM-1+ regulatory B cells in an initial sample from the subject, administering a therapeutic protocol to the subject, and then detecting the number of TIM-1⁺ B cells in a second sample from the subject following administering the therapy. An increase in the number of TIM-1⁺ regulatory B cells, such as CD19⁺TIM-1⁺ regulatory B cells in the second sample as compared to the number of CD19⁺TIM-1⁺ regulatory B cells in the initial sample indicates that the immunosuppressive agent is effective in treating the subject. An decrease or no statistically significant difference in the number of TIM-1⁺ regulatory B cells, such as CD19⁺TIM-1⁺ regulatory B cells in the second sample as compared to the number of TIM-1⁺ regulatory B cells, such as CD19+TIM-1+ B cells in the initial sample indicates that the immunosuppressive agent is not effective in treating the subject. In another example, the TIM-1+ B cell number (or ratio of TIM-1+ to TIM-1− B cells) may be used to monitor response to therapy with an agent aiming to augment the immune response to treat cancer or an immunodeficiency disease. In this case, a decrease in TIM-1+ B cells (or ratio) would indicate effectiveness of the therapy.

The disclosure is illustrated by the following non-limiting Examples.

### EXAMPLES

It is disclosed herein that in vivo, TIM-1 is predominantly expressed on B cells rather than T cells, both constitutively and after activation. Surprisingly, the tolerogenic effects of the low affinity anti-TIM-1 mAb, RMT1-10, on allograft survival are completely dependent on TIM-1+ B cells. TIM-1 identifies a large majority of splenic B cells capable of IL-10 expression regardless of other marker expression. Finally, TIM-1+ Breg are induced by TIM-1 ligation with anti-TIM-1 and can transfer long-term acceptance of islet allografts to otherwise untreated recipients in an IL-10 dependent fashion. Thus, TIM-1 identifies IL-10-expressing Breg across multiple B cell subpopulations.

### Example 1

### Methods

Mice. The following animals were used: Sex-matched 6-10 week old C57BL/6 (H-2b) and BALB/c (H-2d) mice (National Cancer Institute) and IL-4^{-/-}, IL-4Rα^{-/-}, and IL-10^{-/-} BALB/c mice (The Jackson Laboratory) were used. BALB/c IL-4 reporter mice (4get), C57BL/6 IL-10 GFP reporter (Tiger), B-deficient JHD (BALB/c) mice, and IL-10^{-/-} (BALB/c) mice also were utilized. All animals were housed under specific-pathogen-free conditions.

In vivo treatment protocols. α-TIM-1 mAb RMT1-10 (rat IgG2a) was administered i.p. on days −1 (0.5 mg), 0 and 5 (0.3 mg each), relative to day of transplantation. As indicated, BALB/c mice were treated with anti-murine CD20 mAb 18B12 (IgG2a), 250 μg (i.v.) on days −14 and −1 relative to transplantation. This mAb (Biogen-IDEC Pharmaceuticals, San Diego, Calif.), depletes >95% of B cells in the circulation, spleen, and LN, and 86% of mature and immature B cells in the marrow for 2-3 weeks (Hamel et al., 2008).

Islet isolation and transplantation. Islets from C57BL/6 donors were digested with Collagenase V (Sigma), purified by filtration through a 100-um nylon cell strainer (BD Biosciences), hand-picked under a stereomicroscope, and placed (400 islets/recipient) under the left renal capsule treated BALB/c and JHD recipients with streptozocin-induced diabetes, as we described (Ariyan et al., 2003; Salvalaggio et al., 2002). All recipients had glycemia <150 mg/dl by day 2 post-transplant. Blood glucose >250 after engraftment was defined as rejection.

Flow cytometry. Biotin or fluorochrome-conjugated mAbs were purchased from BD Biosciences or eBioscience, except for α-TIM-1-PE (Biolegend). Flow acquisition was performed on LSR11 or FACSCalibur analyzers (BD Biosciences), and data were analyzed using Flowjo software (Tree Star). Background staining was determined with isotype-matched controls. Where IL-4 and IL-10 were detected by GFP reporter expression, cells from wt littermates were used as negative controls. For detection of intracellular cytokines: T cells were cultured for 4 hrs with PMA (50 ng/ml; Sigma), ionomycin (500 ng/ml; Sigma), and GolgiPlug (1 μl/ml, BD Biosciences); B cells were cultured for 5 hrs with LPS (10 ug/ml), PMA (50 ng/ml; Sigma), ionomycin (500 ng/ml; Sigma), and GolgiPlug (1 μl/ml; BD Biosciences), as described (Yanaba et al., 2008a). Intracellular staining was conducted using intracellular staining kits from BD Biosciences or e-Biosciences.

Cell preparation and adoptive transfer. CD19⁺ B cells were enriched by negative selection (EasySep; StemCell Technologies). Purity of the B-cell subset was over 95%. CD19⁺TIM-1⁺ and CD19⁺TIM-1⁻ B cells were subsequently isolated by FACs using a BD FACSAria. For adoptive transfer studies, 1×10⁷ sort-purified CD19⁺, CD19⁺TIM-1⁻, and or CD19⁺TIM-1⁺ B cells from spleens of naïve BALB/c mice or BALB/c recipients of C57BL/6 islets (day 14) were injected (i.v.) into fresh JHD allograft recipients.

Statistics Statistical analyses used unpaired Student's t test and Log-rank (Mantel-Cox) test. Differences were considered to be significant at p<0.05.

### Example 2

### B Lymphocytes Express High Levels of TIM-1 Compared to T Cells

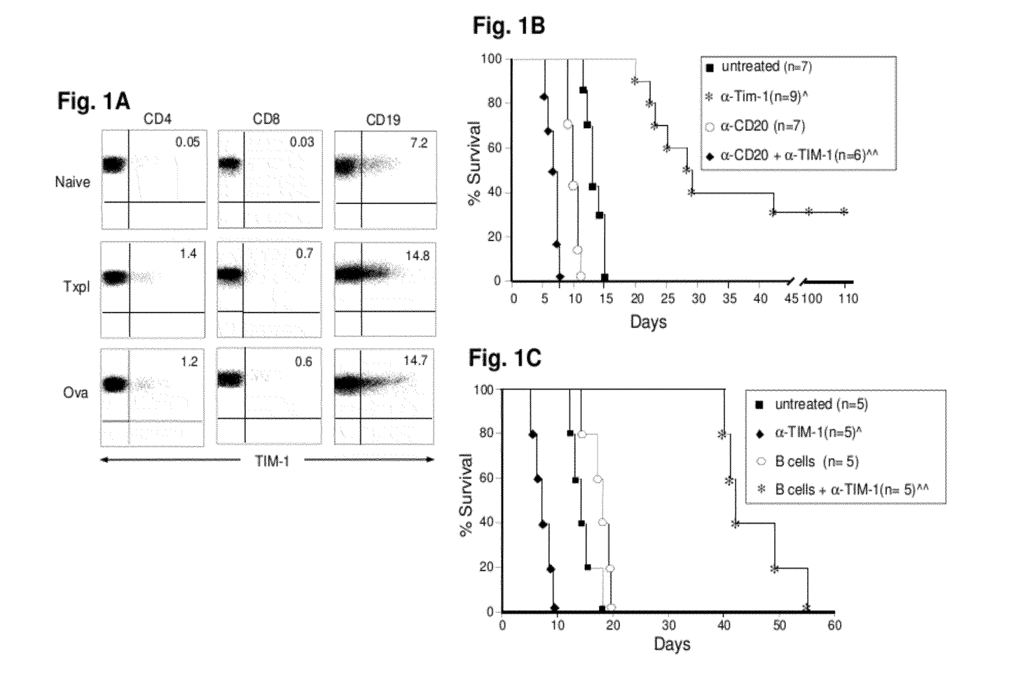

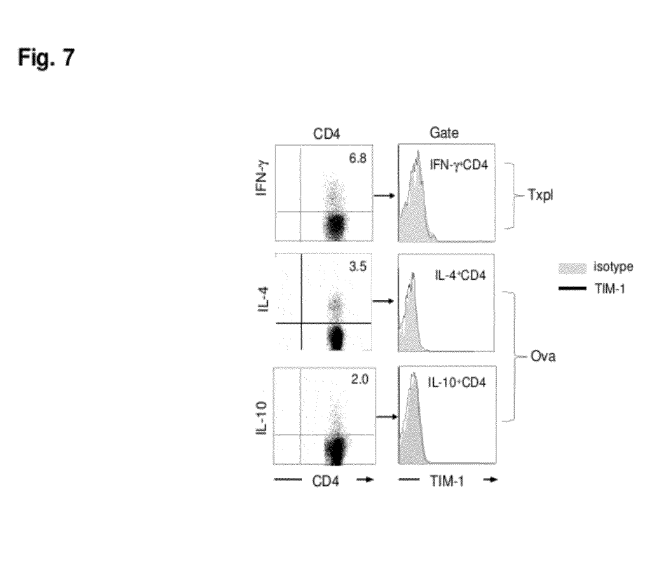

Previous reports have demonstrated that TIM-1 is expressed by activated T cells and in particular, on polarized Th2 cells in vitro (Umetsu et al., 2005). However, in vivo, it was determined that <2% of splenic CD4 cells expressed TIM-1, even after immunization with an islet allograft or ovalbumin (FIG. 1A). This was not increased by gating on Th1 (IFNγ+) or Th2 (IL-4+ or IL-10+) CD4 cells induced by these stimuli (FIG. 7). Similarly, TIM-1 expression on Foxp3+ Treg, and CD11c⁺ and CD11b⁺ leukocytes was minimal. In marked contrast, it was found that in naive mice, between 5-8% of splenic B cells constitutively express TIM-1 (FIG. 1A) and this increases to 10-15% after immunization with an allograft or ovalbumin. Since splenic B cell number did not vary significantly with immunization, the increased percentage of TIM-1+ B cells directly reflects an increase in the number of B cells expressing TIM-1.

### Example 3

### B Lymphocytes are Required for Prolonged Allograft Survival Induced by Anti-TIM-1

Based on high levels of expression, the role of TIM-1 on B cells was addressed. Chemically diabetic BALB/c recipients of C57BL/6 islet allografts were untreated or treated with anti-TIM-1 (RMT1-10). It was previously demonstrated that this low affinity anti-TIM-1 mAb, prolongs cardiac allograft survival (Ueno et al., 2008). In untreated wt mice, islet rejection occurred with a median survival time (MST) of 12 days (FIG. 1B). Treatment with 3 doses of anti-TIM-1 significantly prolonged islet allograft survival (mean survival time (MST) 28 days (d)) with ˜30% of mice achieving long-term engraftment (>100d). Depletion of B cells in recipients prior to transplantation using anti-CD20, slightly shortened allograft survival compared to B cell-intact mice (MST 10d). Surprisingly, rather than prolonging allograft survival, anti-TIM-1 treatment significantly accelerated allograft rejection in B depleted recipients (MST 5.5d). This was confirmed in chemically diabetic B cell deficient JHD (BALB/c) recipients of C57BL/6 islets. Untreated JHD recipients reject islet allografts with a MST of 14d (FIG. 1C). Like B cell depleted wt recipients, JHD mice treated with anti-TIM-1 exhibit a two-fold acceleration in rate of acute rejection (MST 7d). Reconstitution of JHD mice with 10⁷ wild-type (wt) B cells, followed by anti-TIM-1 treatment, significantly enhanced allograft survival (MST 42d). Thus, B cells are not only required for enhanced allograft survival by anti-TIM-1, but in their absence, this monoclonal antibody (mAb) paradoxically shortens survival. This reveals a key role for B cells in TIM-1-mediated allograft survival.

### Example 4

### B Cells are Required for Th2 Cytokine Expression Induced by Anti-TIM-1

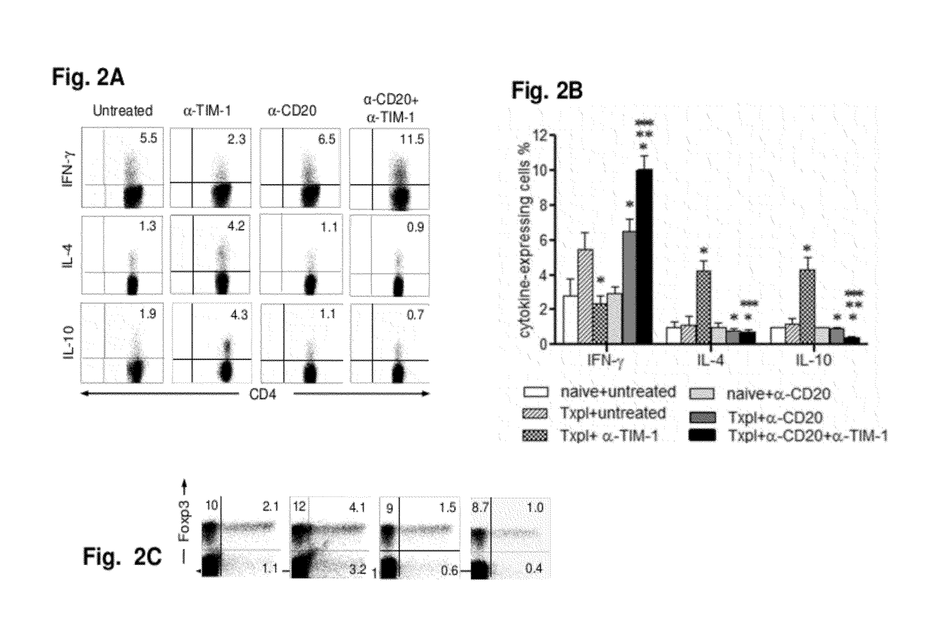

Previous results showed that anti-TIM-1 inhibited Th1 and augmented Th2 responses, and that this was required for prolonged cardiac allograft survival (Ueno et al., 2008). Similarly, in islet allograft recipients, anti-TIM-1 inhibited IFNγ and augmented IL-4 and IL-10 expression by CD4 cells (FIGS. 2A and 2B). B cell depletion alone (anti-CD20) modestly increased IFNγ expression in allograft recipients. However, treatment of B cell depleted recipients with anti-TIM-1 significantly enhanced IFNγ and completely prevented the increase in Th2-cytokines normally observed after anti-TIM-1 treatment. Thus, B cells are required for the Th2 shift observed after anti-TIM-1 treatment. Previous findings also suggest that anti-TIM-1 can enhance Treg function (Ueno et al., 2008). This may be explained in part by enhanced IL-10 expression by Foxp3+ Treg observed after anti-TIM-1 treatment, averaging 1.6-fold (FIG. 2C). However, in the setting of B cell depletion, anti-TIM-1 significantly reduced IL-10 expression on Treg by over 40%, suggesting that B cells also support Treg function after TIM-1 ligation with RMT1-10. IL-10 expression after anti-TIM-1+ B cell depletion was reduced 40% as compared to B cell depletion alone and 3-fold from anti-TIM-1 without B cell depletion.

### Example 6

### Treatment of Immune Mediated Disease by Administration of TIM-1+ Regulatory B Cells

Human PBMC are collected by peripheral venipuncture or apheresis from patients with autoimmune disease or from patients who have received an organ or cellular transplants (allografts). For example, approximately 1 million PBMC can be obtained per ml of human blood. More B cell can be obtained by apheresis, a procedure that can easily result in hundreds of millions of human PBMC in a few hours with low risk. B cells are enriched by incubation of PBMC with anti-CD19 followed by immunomagnetic beads, such as the EASAYSTEP™ CD19 positive selection kit (Stem Cell Technologies, Vancouver, Canada). The TIM-1+ fraction of B cells is isolated by fluorescent activated cell sorting (FACs) either prior to or after stimulation and in vitro expansion. Between 2−5×10⁴ B cells are purified per million PBMC (2-5% of PBMC). From 50 ml of blood, 2×10⁶ total B cells or 4×10⁵ TIM-1+ B cells is recovered.

B cells are expanded through CD40 ligation provided by soluble CD154 (RnD Systems) or by NIH 3T3 fibroblast cell lines stably transfected to express human CD 154. A calcineurin inhibitor is added initially to inhibit growth of contaminating T cells. rIL-4 (2 ng/ml) is added as a growth factor. Stimuli are replaced every 3-4 days. B cells are expanded over many weeks resulting in over 10¹⁰ cells. In some embodiments, CD154 stimulation is combined with (or replaced by) B cell receptor ligation (such as with anti-IgM antibody, for example 0.5 ug/ml) which along with rIL-4 augments TIM-1 and IL-10 expression by TIM-1+ Bregs.

In some examples, if TIM-1+ cells are selected initially, no further selection is necessary after ex vivo expansion. In other examples, if whole B cells are used, TIM-1+ B cells are selected after in vitro expansion to enrich for regulatory B cells, as above, by FACs or immunomagnetic bead selection.

TIM-1+ B cells are infused back into the patient. The number of such cells required are based on extrapolation from preliminary studies in animal models. For example, these include studies in immunodeficient mice (SCID common γ-chain knockout mice, repopulated with autologous human blood cells and then receiving as allogeneic cell transplant (such as human islets). Alternatively, nonhuman primates can be used as allograft recipients and the effects of infused TIM-1+ autologous B cells examined for their ability to inhibit rejection of islets or a kidney transplant.

For one example, 10⁹ or 10¹⁰ autologous TIM-1+ B cells are infused into an allograft recipient or a patient with autoimmune disease or an allergic condition. These cells suppress immune system reactivity and limit requirements for other immunosuppressive agents. In a variation, other markers (such as immature transitional B cells, or CD1dHiCD5+ B cells) can be initially employed to enrich for human Breg prior to in vitro expansion. Then, after expansion, Bregs are further enriched by isolation of TIM-1+ B cells. Data shows that within other B cell subsets, TIM-1 identifies those B cells that have regulatory activity, based on IL-10 expression.

In other embodiments, B cells are expanded in vitro in response to specific antigens involved in the immune response being treated. For example, in transplant patients, B cells are exposed to allogeneic antigen presenting cells in addition to rIL-4 and possibly CD154 costimulation. This promotes expansion of antigen-specific B cells are more effective and do not require as much expansion of B cells that have truly polyclonal specificity.

The infused TIM-1+ B cells are monitored in the peripheral blood by surface phenotyping (immunofluorescence) or by quantitative PCR, to determine when retreatment is necessary.

### Example 7

### Promoting the Immune Response to Treat Patients with Cancer of Infectious Diseases by Targeting TIM-1

Immunodeficiency states are characterized by defects in tumor surveillance and fighting off infections. On the other hand, certain infections and malignancies have evolved ways to evade, exhaust, or directly attack the immune response, resulting in generalized or localized immunodeficiency. In each of these settings, inhibiting endogenous suppressive mechanisms, can boost the immune response and promote immune responses against chronic viral infections or tumors. Bregs promote tolerance to a wide variety of immune responses, and their elimination promotes immune responsiveness.

Patients with diseases caused by or leading to immunodeficiency are treated by depletion of B cells expressing TIM-1. Since B cells are the main population of human lymphocytes expressing TIM-1, treatment of patients with anti-TIM-1 mAbs or immunotoxin conjugates primarily depletes TIM-1+ B cells, a population that down-regulates and inhibits immune reactivity. Targeting a single molecule is a significant advantage over trying to target cells that express a combination of targets, each of which is also expressed by cells that one does not wish to deplete.

Patient blood is first examined by flow cytometry to document the expression of TIM-1 on B cells versus other leukocytes, and the proportion of total B cells expressing TIM-1 is determined. A depleting anti-TIM-1 mAb is administered intravenously. The mAb can be humanized to lessen immunogenicity and improve half-life resulting in a more sustained depletion of TIM-1+ B cells. Numerous mAbs are currently used for depletion or blockade in human subjects; similar does are used for TIM-1 antibodies. Exemplary does are two doses of 30 mg or two doses of 1000 mg each. Patients are monitored for depletion of TIM-1+ B cells in peripheral blood and therapy re-administered when TIM-1+ B cells begin to re-emerge. Anti-TIM-1 mAbs block the inhibitory function of regulatory B cells and stimulate their effector function (for example, IFNγ production) leading to augmented B and T cell responses against pathogens or tumors.

### Example 5

### B Cell IL-4 and IL-10 are Primarily Expressed by the TIM1+ Subset and are Induced by TIM-1 Ligation

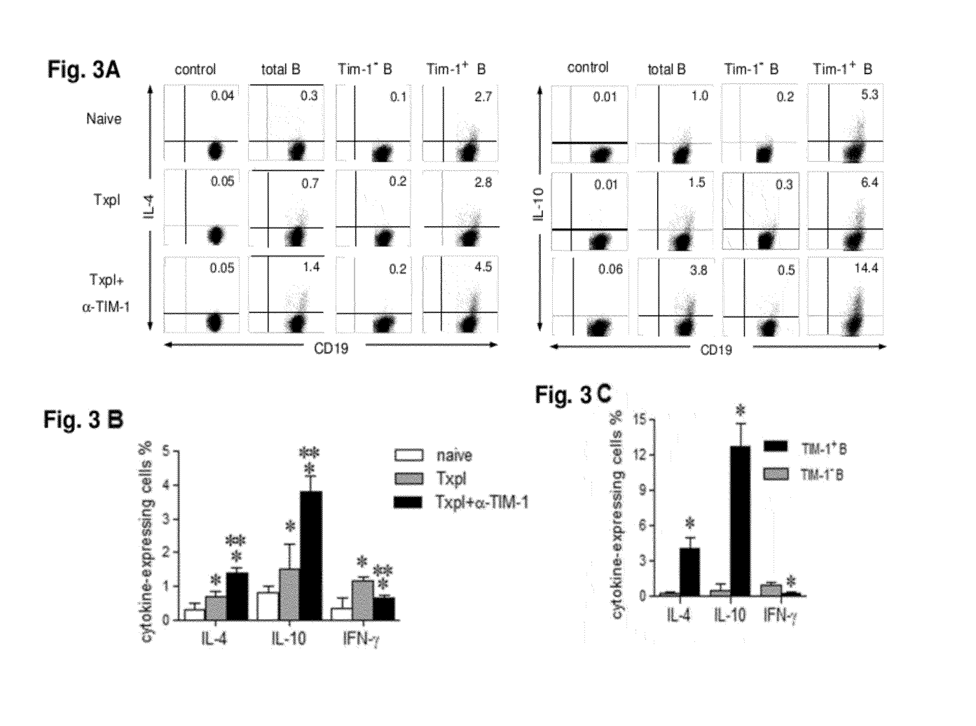

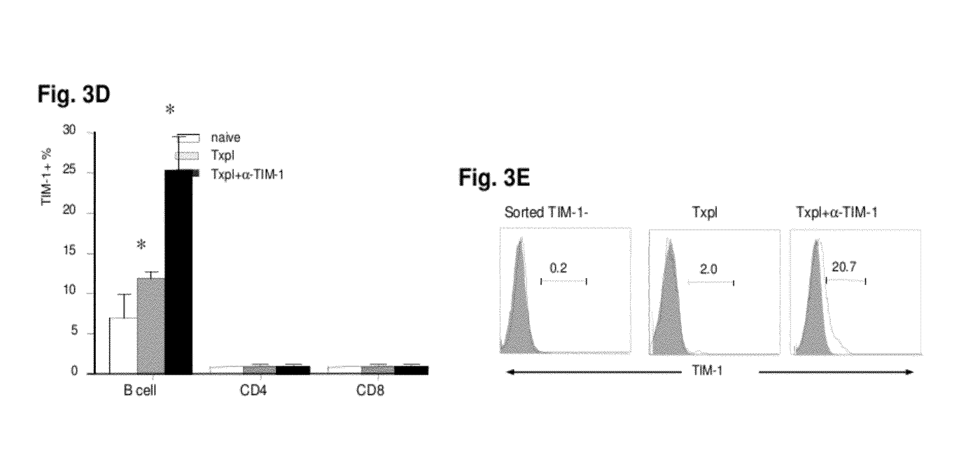

Anti-TIM-1 enhances Th2 cytokine expression in the presence of B cells, yet promotes expression of Th1 cytokines in the absence of B cells. Cytokines produced by B cells can influence T cell polarization (Harris et al., 2000). This raises the question as to whether TIM-1 ligation augments IL-4 production by B cells, which could then promote a Th2 response. Using IL-4 EGFP reporter mice (BALB/c) and wt littermates as negative controls, very low but reproducible IL-4 expression by splenic B cells from naïve mice was detected (FIG. 3A). Immunization by islet transplantation increased IL-4 expression from 0.2 to 0.8%. This doubled when recipients received anti-TIM-1 (FIGS. 3A and 3B). IL-4 expressing B cells were almost entirely found within the TIM-1+ subset (FIGS. 3A and 3C). Moreover, the percent of TIM-1+ B cells expressing IL-4 increased several-fold when allograft recipients were treated with anti-TIM-1 (FIG. 3A).

In addition to promoting a Th2 response, B cells might support TIM-1-mediated engraftment through regulatory activity. Breg activity corresponds closely with IL-10 expression (Dilillo et al.; Mauri and Ehrenstein, 2008). B cells expressing IL-10 are very infrequent (˜1%) amongst splenic B cells (FIGS. 3A and 3B). While islet transplantation increased IL-10 expression on B cells from 0.8% to 1.5%, treatment of recipients with anti-TIM-1 increased IL-10 expression on B cells almost five-fold (˜4%; FIG. 3B). As with IL-4, TIM-1+ B cells were highly enriched for IL-10 expression. In naïve mice, and in untreated allograft recipients, roughly 5% of TIM-1+ B cells express IL-10, a 25-fold enrichment compared to TIM-1⁻ B cells and >5-fold enrichment compared to total splenic B cells (FIG. 3A). Treatment of allograft recipients with anti-TIM-1 increased TIM-1⁺ B cells expressing IL-10 more than two-fold (˜13%; FIG. 3C). Thus, TIM-1+ B cells are highly enriched for IL-4 and IL-10 expression and cytokine expression is augmented by anti-TIM-1 treatment. Since treatment did not affect splenic B cell number, these percentages reflect an increase in number of B cells expressing these cytokines.

Consistent with FIG. 1A, transplantation increases the frequency of TIM-1⁺ B cells almost two-fold, reaching 12-15% of splenic B cells (FIG. 3D). Treatment of transplant recipients with anti-TIM-1 further increases TIM-1 expression averaging 25%, and up to 50% of splenic B cells in some mice. The increase in TIM-1 expression on B cells following TIM-1 ligation appears relatively specific since other surface molecules including Class II and costimulatory ligands such as CD80, CD86, PD-L1, PD-L2, and CD40, were unchanged.

To determine whether anti-TIM-1 can induce TIM-1 expression de novo, sort-purified TIM-1⁻ B cells were transferred into JHD allograft recipients with or without anti-TIM-1 treatment. B cell TIM-1 expression remained low (˜2%) 14 days after transfer into untreated JHD allograft recipients (FIG. 3E). However, treatment of allograft recipients with anti-TIM-1 induced TIM-1 expression on ˜20% of B cells that were originally TIM-1⁻. Taken together, anti-TIM-1 both increases the number of TIM-1+ B cells and increases the number of TIM-1+ B cells expressing IL-4 and IL-10. Thus, anti-TIM-1 induces a subset of TIM-1⁺ B cells with potential regulatory activity (see FIG. 3).

### Example 6

### Anti-TIM-1 Mediated Graft Survival is Associated with B Cell TIM-1 Expression, IL-10 and IL-4 Signaling

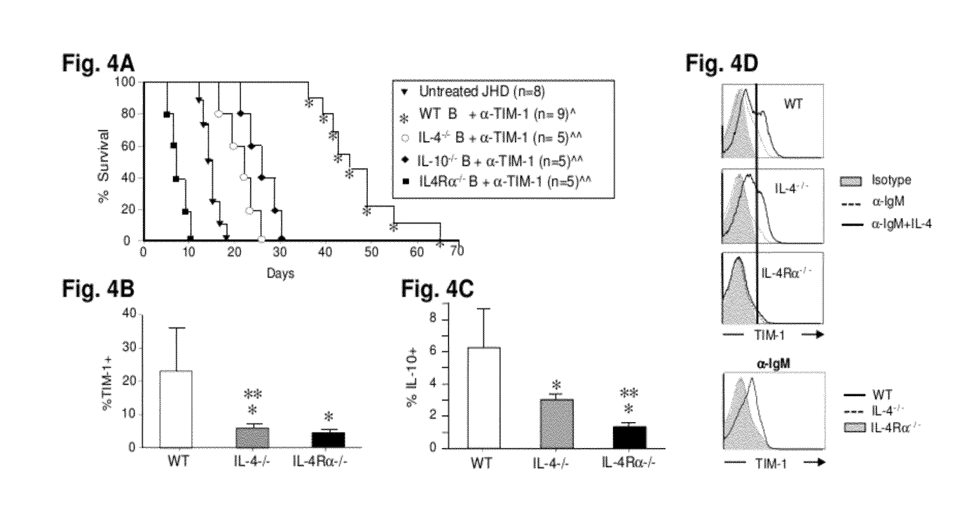

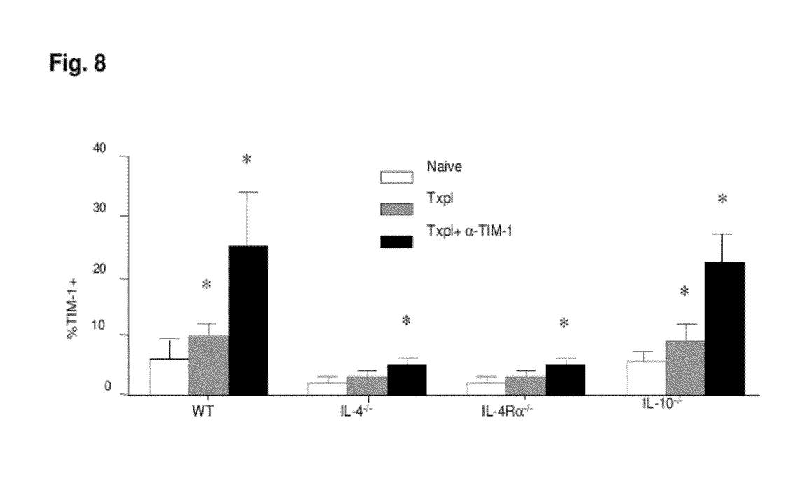

The role of IL-4 and IL-10 expression by B cells on their immunomodulatory function was assessed. Anti-TIM-1 shortens allograft survival in B cell deficient (JHD) allograft recipients, but significantly enhances allograft survival when recipients are reconstituted with wt B cells (FIGS. 1C and 4A). In contrast, reconstitution of anti-TIM-1-treated JHD recipients with IL-4−/− or IL-10−/− B cells only partially restores allograft survival (MST 23d and 26d, respectively) compared to wt B cells (MST 46d; FIG. 4A). This suggests that IL-10 and IL-4 are both required for immunomodulatory function of B cells mediated through TIM-1. However, while IL-10−/− mice had normal B cell TIM-1 expression, IL-4−/− mice exhibited a marked reduction in TIM-1+ B cells both constitutively, and after transplantation (FIG. 8). Indeed, after adoptive transfer into anti-TIM-1 treated JHD allograft recipients, over 20% of wt B cells express TIM-1 and >6% express IL-10, whereas only 6% of IL-4−/− B cells express TIM-1 and 3% express IL-10 (FIGS. 4B and 4C). The defects in TIM-1 and IL-10 expression were even more severe in B cells from IL-4R−/− mice. After adoptive transfer into treated JHD recipients, only 3% of IL-4R−/− B cells expressed TIM-1 and 1.3% expressed IL-10 (FIGS. 4B and 4C). Moreover, IL-4R−/− B cells were unable to protect JHD recipients from accelerated rejection after anti-TIM-1 treatment, as seen in mice lacking B cells altogether (FIGS. 1C and 4A). Thus, prolongation of anti-TIM-1-mediated graft survival by B cells correlates directly with TIM-1 and IL-10 expression, and these both depend on IL-4 signaling.

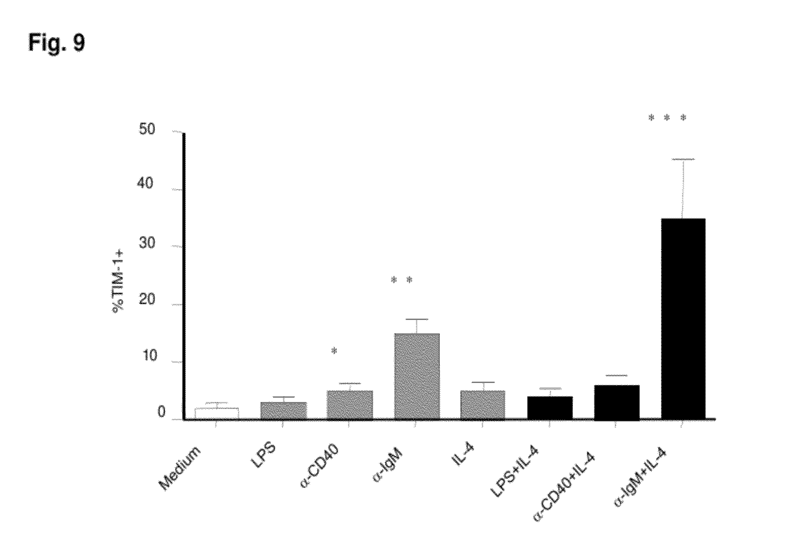

To further confirm the role of IL-4 signaling on TIM-1 induction, sort-purified TIM-1⁻ B cells from wt, IL-4−/− and IL-4R−/− mice were stimulated in vitro for 48 hours in the presence or absence of exogenous IL-4. TIM-1 expression was readily induced on wt TIM-1⁻ B cells by B cell receptor ligation with anti-IgM, increasing from 2-3% (media alone) to over 15% (FIG. 4D top histogram and FIG. 9). Moreover, TIM-1 induction by anti-IgM was markedly enhanced by exogenous IL-4, reaching 30% of the total B cell population. In contrast, anti-CD40 only slightly increased TIM-1 expression (4%) and LPS was without effect (FIG. 9). Similarly, IL-4 alone had only a minor effect on TIM-1 expression above media control (4%) and this was not increased by adding anti-CD40 or LPS (FIG. 9). In contrast to IL-4, other Th2 cytokines such as IL-10, IL-5, and IL-13, had no effect on B cell TIM-1 expression induced by anti-IgM, while IL-12 and IFNγ actually reduced TIM-1 expression.

Compared to wt B cells, anti-IgM had a markedly reduced effect on TIM-1 induction in IL-4−/− B cells (FIG. 4D, second and bottom histogram). However, when exogenous IL-4 was added, TIM-1 expression in IL-4−/− and wt B cells was comparable (FIG. 4D top two histograms). Thus, IL-4−/− B cells are capable of TIM-1 induction if IL-4 signaling is provided. In contrast, IL-4R−/− B cells had defective TIM-1 induction after anti-IgM, and as expected, this was not influenced by exogenous IL-4 (FIG. 4D third histogram). These results correlate closely with the in vivo effects of B cells from IL-4−/− and IL-4R−/− mice shown above (FIGS. 4A-4C) and indicate that TIM-1 induction on TIM-1⁻ B cells requires BCR and IL-4 signaling.

### Example 7

### TIM-1⁺B Cells are Regulatory and can Transfer Donor-Specific Tolerance

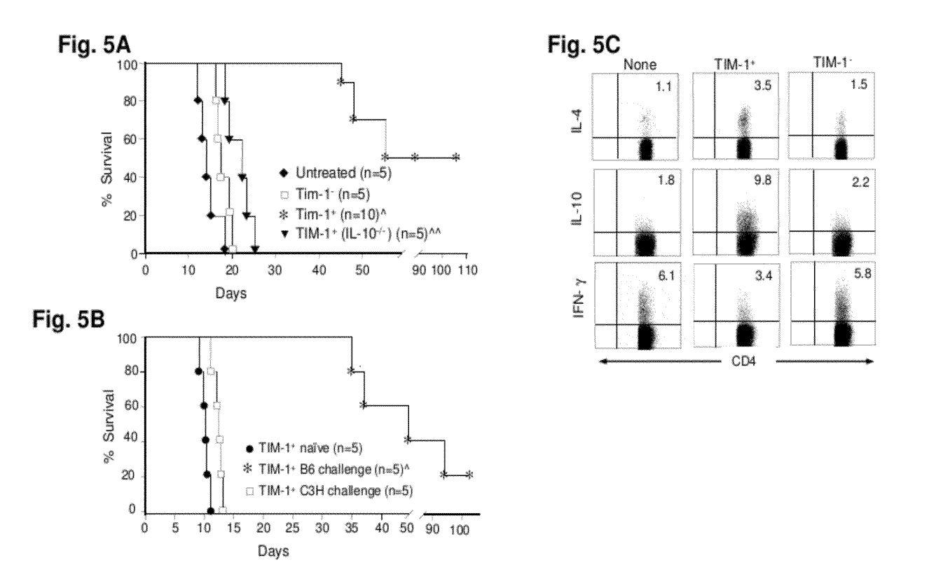

The data above suggest that TIM-1⁺ B cells are themselves regulatory. To directly test this, BALB/c allograft recipients treated with anti-TIM-1 were sacrificed on day 14. Splenic B cells were sorted into TIM-1⁺ and TIM-1⁻ populations and transferred into otherwise untreated JHD recipients of C57BL/6 islets. TIM-1⁺ B cells markedly prolonged allograft survival with 50% of allografts surviving indefinitely (FIG. 5A). In contrast, transfer of TIM-1⁻ B cells was unable to prolong graft survival in otherwise untreated recipients (MST 15d). TIM-1⁺ B cells from IL-10−/− islet allograft recipients were also defective in their regulatory capacity (MST 22d) compared to wt TIM-1⁺ B cells.

Next, it was addressed whether TIM-1⁺ B cells induced in the absence of TIM-1 ligation (which augments TIM-1, IL-10 and IL-4 expression) are also regulatory. TIM-1⁺ B cells obtained from naïve (untransplanted) mice were unable to prolong allograft survival in JHD allograft recipients (FIG. 5B). In contrast, TIM-1⁺ B cells from untreated BALB/c recipients of a C57BL/6 allograft were able to markedly prolong graft survival in JHD recipients of C57BL/6 islets. These data are consistent with previous reports that Breg must be activated to be effective (Dilillo et al., 2010). However, Breg activity appears antigen-specific, since TIM-1⁺ B cells from mice receiving an unrelated (C3H) allograft were unable to prolong graft survival when transferred into JHD recipients of C57BL/6 islets. Thus, TIM-1⁺ B cells exhibit potent regulatory function. In this regard, the transfer of TIM-1⁺ but not TIM-1⁻ B cells, augments IL-4 and IL-10 and inhibits IFNγ expression by endogenous CD4 cells (FIG. 5C).

### Example 8

### TIM-1 Defines Breg that Encompass Other IL-10-Expressing Breg Sub-Populations

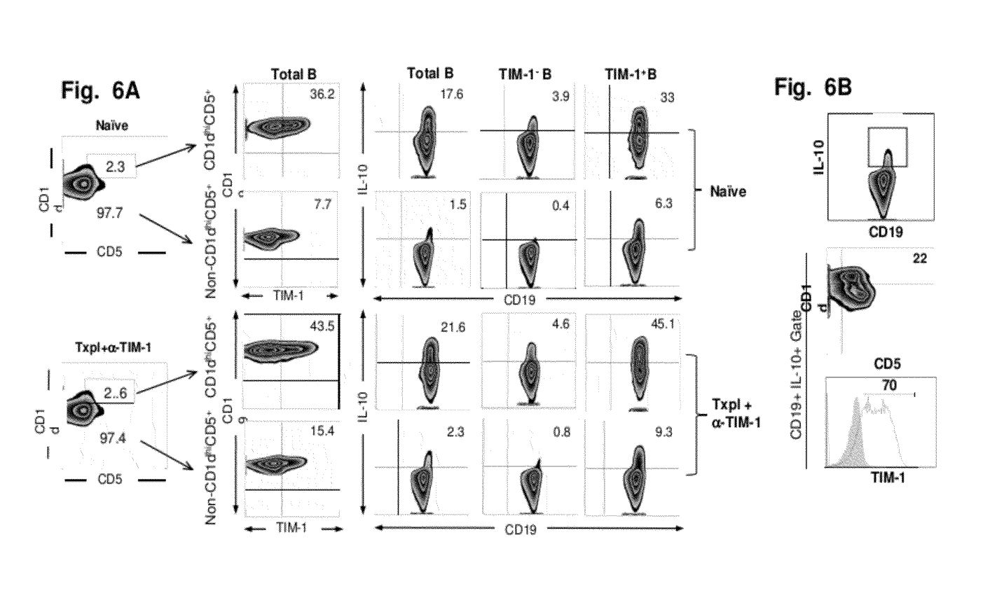

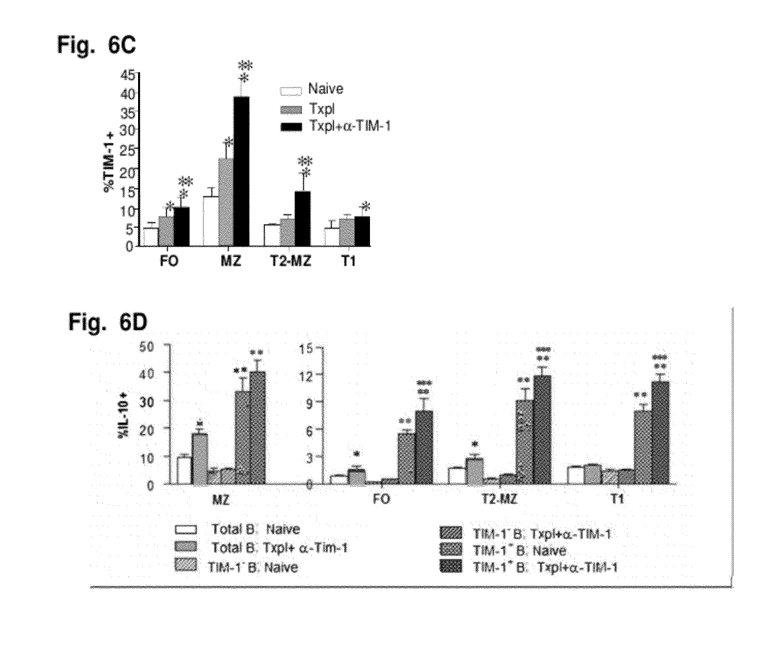

Breg, defined by their functional capacity and ability to produce IL-10, have been reported as low frequency cells belonging to various B cell subpopulations. Recently, B cells expressing a CD1dHiCD5+ phenotype were reported to be highly enriched for IL-10 expression and encompass the vast majority of Bregs (Dilillo et al., 2010). CD1dHiCD5+ cells make up only ˜2% of B cells, but are highly enriched for IL-10 expression (17%; FIG. 6A). However, the 36% of CD1dHiCD5+ B cells expressing TIM-1 are even more highly enriched for IL-10 (30-40%). In contrast, TIM1⁻ cells within the CD1dHiCD5+ subset exhibit an ˜8-fold decrease in frequency of IL-10 expression (FIG. 6A). On a numerical basis, TIM-1 identifies >82% of Breg within this subset in naïve mice.

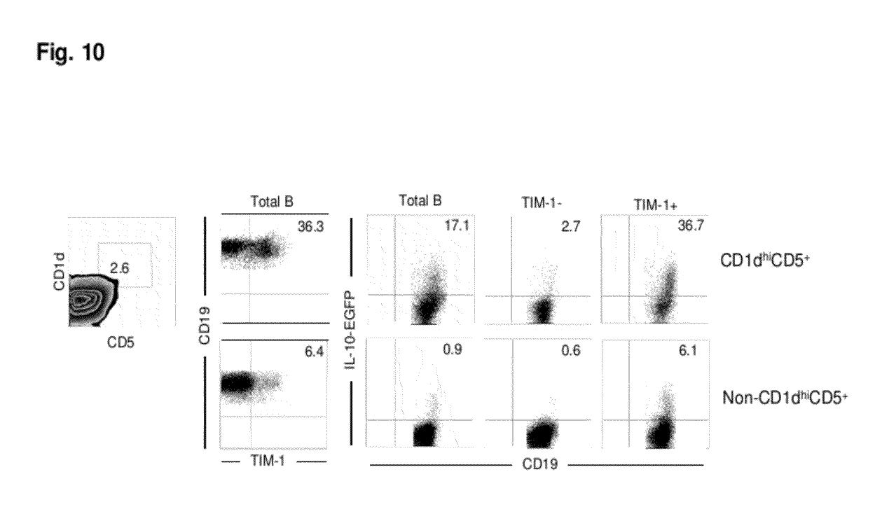

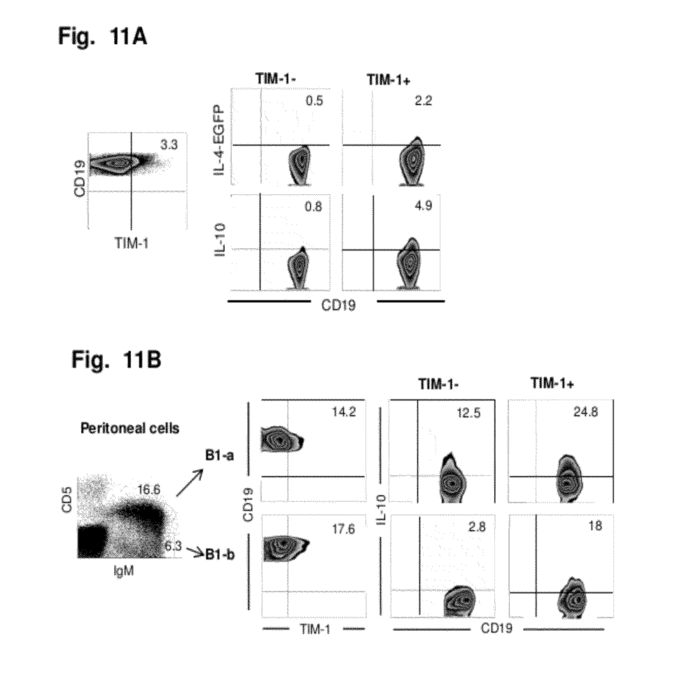

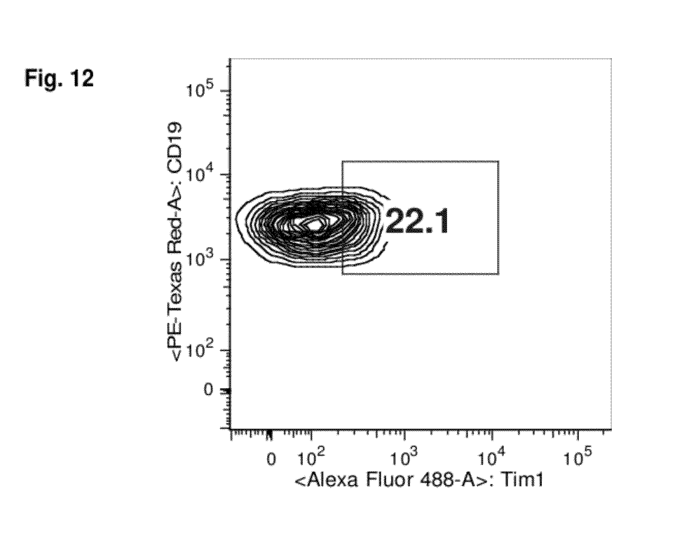

While the CD1dHiCD5+ population is enriched for IL-10 expression, ˜98% of B cells fall outside of this region. Even though IL-10 is expressed by only 1.5% of the non-CD1dHiCD5+ population, numerically, these comprise a majority of IL-10 expressing B cells in spleen (FIG. 6A). However, there has been no consistent marker allowing identification of these rare cells within this vast B cell population. It is demonstrated herein that TIM-1 identifies 8% of non-CD1dHiCD5+ B cells and these are >15-fold enriched for IL-10 expression (6%) compared to their TIM-1-counterparts. Similar findings were observed using IL-10 reporter mice (C57BL/6 background), confirming the findings in a second strain (FIG. 10).

In allograft recipients, TIM-1 ligation increases the percent of CD1dHiCD5+ B cells expressing TIM-1 from ˜35% to ˜45%, and the percent of TIM-1+ cells expressing IL-10 reaches 45% (FIG. 6A). In the majority non-CD1dHiCD5+ B cell population, transplantation and TIM-1 ligation doubles B cells expressing TIM-1 (15%), with 9-10% of these now expressing IL-10. Thus, TIM-1 identifies most IL-10+ B cells within the CD1dHiCD5+ subset and also identifies a large number of B cells expressing IL-10 that are excluded by the CD1dHiCD5+ phenotype. Moreover, TIM-1+ Breg are induced by TIM-1 ligation in the presence of antigenic stimulation. To confirm this, we gated on CD19+IL-10+ B cells and then compared the percent that were CD1dHiCD5+ versus TIM-1+. In these experiments, ≦25% of all IL-10+ B cells in spleen fell within the CD1dHiCD5+ gate (FIG. 6B). In contrast, TIM-1 identifies ˜70% of all IL-10+ B cells.

The distribution of TIM-1 on typical splenic B cell subsets was examined in naïve mice (Tung et al., 2004). TIM-1 was preferentially expressed on marginal zone (MZ) B cells (15%), but is also expressed on 5-7% of immature transitional 1 (T1), transitional 2-marginal zone (T2-MZ) precursors and follicular (FO) B cells (FIG. 6C). In each subset, TIM-1 identifies B cells that are 7-20 fold enriched for IL-10 expression compared to their TIM-1− counterparts (FIG. 6D). Finally, within each B cell subset, TIM-1 ligation in the presence of alloantigen significantly enhanced the percent of cells expressing TIM-1 and/or the percent of TIM-1⁺ B cells expressing IL-10. For example, amongst MZ B cells 33% of TIM-1⁺ cells express IL-10. IL-10 expression in this subset increases to 40% after anti-TIM-1 treatment of allograft recipients. However, the number of TIM-1⁺ MZ B cells increases 2.8-fold (from 14% to 40%; FIG. 6C), resulting in an overall 3.5-fold increase in number of TIM-1+ IL-10+ MZ B cells. FOB cells comprise ˜40% of splenic B cells of which <1% express IL-10 (FIG. 6D). However, ˜6% of TIM-1+FOB cells express IL-10, a >20-fold increase from their TIM-1⁻ counterparts. Moreover, TIM-1 ligation in transplant recipients increases the number of TIM-1⁺ FO B cells over 2.2-fold and increases IL-10 expression on TIM-1+ B cells by 45% resulting in a >3-fold increase in TIM-1⁺IL-10⁺ B cells in this large subset (FIGS. 6C and 6D). A similar increase in TIM-1+ IL-10+ cells is observed in MZ-T2 and T1 subsets. Thus, anti-TIM-1 treatment of allograft recipients increase the percent of B cells expressing TIM-1 and percent of TIM-1+ cells expressing IL-10 within each subset. Thus, TIM-1 identifies B cells that are highly enriched for IL-10 production regardless of the other phenotypic markers expressed by these regulatory B cells.

TIM-1 has been identified as a T cell costimulatory molecule that regulates both autoimmunity and alloimmunity through its effects on CD4 effector responses (Kuchroo et al., 2008). The data presented herein shows that TIM-1 plays an important functional role on B cells that is critical for anti-TIM-1 mediated tolerance. Thus, interference with TIM-1 using an anti-TIM-1 mAb (RMT1-10) that normally promotes a Th2 response and prolongs allograft survival, paradoxically induces a Th1 response and actually shortens allograft survival in the absence of B cells. TIM-1 ligation of B cells enhances their IL-4 and IL-10 expression and B cell IL-4 is required for the Th2 deviation normally observed after anti-TIM-1 treatment. In this regard, Harris et al previously demonstrated that B cell IL-4 can enhance Th2 responses (Harris et al., 2000). While very few B cells express IL-4, it is shown herein that these are greatly enriched in the fraction bearing TIM-1. Notably, over-expression of TIM-1 in T cells augments IL-4 transcription, suggesting some commonality in signaling (de Souza et al., 2005). In addition to promoting Th2 deviation, TIM-1 ligation enhances IL-10 expression on Treg in a B cell dependent manner. It was previously showed that Treg and Th2 deviation are both required for prolonged allograft survival induced by anti-TIM-1 (Ueno et al., 2008).

Breg are now recognized to contribute towards tolerance in a number of autoimmune models, where depletion of B cells enhances rather than decreases immune reactivity (Dilillo et al., 2010; Mauri and Ehrenstein, 2008; Mizoguchi and Bhan, 2006; Yanaba et al., 2008b). Such regulatory activity can be found in various B cell subsets and is IL-10 dependent. Only 1-2% of all B cells in spleen and even less in lymph node and bone marrow are “IL-10 competent” (Yanaba et al., 2008b). These factors have complicated the identification and study of Breg. The results presented herein provide a unique subset of B cells with regulatory function.

CD1dHiCD5+ B cells exhibit a higher frequency of IL-10 expression than previously described B cell subsets. Moreover, this subset was purported to identify most IL-10-producing Breg including many Breg previously attributed to MZ or T2-MZ subsets (Yanaba et al., 2008a; Yanaba et al., 2008b). However, the CD1dHiCD5+ population fails to account for a majority of splenic B cells capable of IL-10 production (FIGS. 6A and 6B). In contrast, TIM-1 expression uniquely identifies B cells that express IL-10 regardless of other markers. Thus, within the total B cell population, or within other phenotypic subsets of B cells, TIM-1 identifies those cells that are capable of regulatory function. This is true even within the CD1dHiCD5+ population, where the TIM-1+ B cells comprise a subpopulation highly enriched for IL-10 and regulatory cell activity (>80% of regulatory B cells are contained in this subset) compared to the CD1dHiCD5+TIM-1− population which contains few regulatory cells.

It is demonstrated herein that TIM-1⁺ B cells are enriched 8 to 20 fold for cells expressing IL-10 compared to their TIM-1⁻ counterparts. Moreover, unlike other markers (Dilillo et al., 2010), TIM-1 can also identify B cells enriched for IL-10 in lymph nodes. For example, in naïve mice, TIM-1 identifies a 3-5% of B cells in LN that are 6-fold enriched for IL-10 expression. TIM-1 also identifies ˜15% of peritoneal B cells of which ˜20% and 25% of B1-b and B1-a express IL-1. Thus, TIM-1 constitutes the broadest and most specific marker for Breg yet identified.

The finding that TIM-1 ligation on B cells induces a TIM-1+ B cell population with regulatory activity is novel. TIM-1 ligation is currently unique in its ability to induce Breg across a wide spectrum of phenotypes. BAFF and anti-CD40 are reported to induce Breg within the splenic MZ and T2 populations, respectively (Blair et al., 2009; Yang et al.). However, these agents both exhibit dual agonist and antagonist functions raising the concern that they may not uniformly inhibit B cells or the immune response in all disease settings.

In addition to augmenting the number of TIM-1+ B cells, TIM-1 ligation also augmented their ability to express immunomodulatory cytokines (FIG. 4). Thus, TIM-1 ligation in allograft recipients increased TIM-1 expression to over 40% of both CD1dHiCD5+, as well as the larger MZ population, and IL-10 was expressed by 40-45% of these cells. Even within the majority FO B cell population, anti-TIM-1 increased TIM-1 expression 2.4-fold and IL-4 expression almost 2-fold, resulting in a greater than 4-fold increase in Breg.

On the other hand, IL-10 appears to play a more direct role in Breg activity. Although IL-10 deficient B cells have normal TIM-1 expression and induction, they are unable to fully restore anti-TIM-1 mediated graft prolongation in JHD mice and do not transfer tolerance to otherwise untreated allograft recipients.

The increase in TIM-1+ B cells observed in our studies is at least in part due to induction of TIM-1 expression on TIM-1− B cells. While in untreated allograft recipients, TIM-1 is expressed on only 2% of transferred TIM-1− B cells, anti-TIM-1 treatment induces TIM-1 expression on 20% of these B cells. Presumably anti-TIM-1 binds the small population of TIM-1+ B cells or to TIM-1 expressed on other cell types, which in turn enhance B cell TIM-1 expression through conversion and/or proliferation.

Thus, TIM-1 identifies a novel and inclusive marker for Breg that are highly enriched for IL-10 expression in spleen and LN of both naïve mice and those undergoing active immune responses. Transfer of TIM-1+, but not TIM-1−, B cells after antigen challenge markedly prolongs survival of fully MHC mismatched allografts in otherwise untreated recipients. This is the first agent shown to specifically induce Breg in vivo and is the first example of a tolerogenic agent promoting allograft tolerance through Breg.

It will be apparent that the precise details of the methods or compositions described may be varied or modified without departing from the spirit of the described invention. We claim all such modifications and variations that fall within the scope and spirit of the claims below.

1. An isolated population of regulatory B cells, wherein the regulatory B cells are mammalian and wherein the regulatory B cells express T cell immunoglobulin mucin-1 (TIM-1).

2. The isolated population of regulatory B cells of claim 1, wherein the regulatory B cells produce interleukin-10.

3. The isolated population of regulatory B cells of claim 1, wherein the regulatory B cells are CD19⁺CD1d^{high}CD5⁺.

4. The isolated population of regulatory B cells of claim 1, wherein the regulatory B cells are human regulatory B cells.

5. A method of treating a subject with an immune-mediated disorder, or preventing the development of an immune-mediated disorder, comprising administering to the subject a therapeutically effective amount of a composition comprising the isolated population of regulatory B cells of claim 1, thereby treating or preventing the immune mediated disorder in the subject.

6. The method of claim 5, wherein the immune-mediated disorder is inflammation, allegeric disease, graft-versus host disease, transplant rejection, or an autoimmune disorder.

7. The method of claim 6, wherein the immune-mediated disorder is transplant rejection, and wherein the transplant is an organ transplant, bone marrow or other cell transplant, composite tissue transplant, or a skin graft.

8. The method of claim 6, wherein the autoimmune disorder, allergic or inflammatory disorder is multiple sclerosis, inflammatory bowel disease, rheumatoid arthritis, type I diabetes, systemic lupus erythrematosus, contact hypersensitivity, asthma or Sjogren's syndrome.

9. The method of claim 5, wherein the subject is a human.

10. The method of claim 5, further comprising administering to the subject a therapeutically effective amount of an immunomodulatory or an immunosuppressive agent.

11. The method of claim 10, wherein the immunosuppressive agent is a calcineurin inhibitor, an mTOR inhibitor, an antibody, a chemotherapeutic agent irradiation, a chemokine, an interleukins or an inhibitor of a chemokine or an interleukin.

12. A method of treating a subject with a cancer, comprising administering to the subject a therapeutically effective amount of an antibody that specifically binds TIM-1, thereby inducing an immune response to the cancer and treating the subject.

13. The method of claim 12, wherein the subject is a human.

14. The method of claim 13, wherein the antibody is a monoclonal antibody.

15. The method of claim 14, wherein the monoclonal antibody is a humanized antibody or a fully human antibody.

16. The method of claim 12, wherein the cancer is a solid cancer or a hematological cancer.

17. The method of claim 12, further comprising administering to the subject a therapeutically effective amount of a chemotherapeutic agent.

18. A method for assessing the immune status of a subject, comprising selecting a subject with an immune-mediated disorder or suspected of having or being predisposed to an immune-mediated disorder; and detecting the number of CD19⁺TIM-1⁺ regulatory B cells in an initial biological sample from the subject, thereby assessing the immune status of the subject.

19. The method of claim 18, further comprising administering to the subject a therapeutically effective amount of an immunosuppressive agent.

20. The method of claim 18, further comprising detecting the number of CD19⁺TIM-1⁺ B cells in a second sample from the subject following administering the immunosuppressive agent, wherein an increase in the number of CD19⁺TIM-1⁺ regulatory B cells in the second sample as compared to the number of CD19⁺TIM-1⁺ regulatory B cells in the initial sample indicates that the immunosuppressive agent is effective for treating the subject.

21. The method of claim 18, further comprising measuring the ratio of the number of CD19⁺TIM-1⁺ regulatory B cells to CD19⁺TIM-1⁻ regulatory B cells in the sample from the subject.

22. The method of claim 18, wherein detecting the number of CD19⁺TIM-1⁺ regulatory B cells comprises the use of fluorescence activated cell sorting.

23. The method of claim 18, further comprising measuring the production of IL-10 in a sample from a subject, wherein the sample comprises CD19⁺TIM-1⁺ regulatory B cells.

24. The method of claim 18, wherein a higher number of CD19⁺TIM-1⁺ regulatory B cells, or a higher ratio of regulatory to non-regulatory B cells, as compared to a control indicates the subject is immunosuppressed.

25. The method of claim 18, wherein a lower number of CD19⁺TIM-1⁺ regulatory B cells, or a lower ratio of regulatory to non-regulatory B cells, as compared to a control indicates the subject has an immune-mediated disorder.

26. The method of claim 25, wherein the immune-mediated disorder is an inflammatory disorder, allergic disorder, graft-versus host disease, transplant rejection, or an autoimmune disorder.

27. A composition comprising a therapeutically effective amount of isolated regulatory B cells expressing TIM-1 and CD19 in a pharmaceutically acceptable carrier.
